(19) United States
(12) Patent Application Publication
(10) Pub. No.: US 2023/0357363 A1
(43) Pub. Date: Nov. 9, 2023

(54) PROTEIN DOUBLE-SHELL NANOSTRUCTURES AND THEIR USE

(71) Applicant: The Board of Trustees of the Leland Stanford Junior University, stanford, CA (US)

(72) Inventors: Soichi Wakatsuki, Redwood City, CA (US); Chiu Wah, Redwood City, CA (US); Kathleen Miho Sakamoto, Redwood City, CA (US); Kaiming Zhang, Redwood City, CA (US); Naoki Horikoshi, Redwood City, CA (US)

(21) Appl. No.: 18/029,609

(22) PCT Filed: Oct. 5, 2021
(86) PCT No.: PCT/US2021/053541

§ 371 Date: Mar. 30, 2023

Related U.S. Application Data
(60) Provisional application No. 63/088,296, filed on Oct. 6, 2020.

(51) Int. Cl. C07K 14/79; C12N 15/86

(52) U.S. Cl. CPC C07K 14/79; C12N 15/86; C07K 2319/735; C07K 2319/24

(57) ABSTRACT

Protein double-shell nanostructures comprising apoferritin for carrying cargo proteins of interest are provided. Such nanostructures can be used to increase rigidity of a cargo protein of interest to allow structures of small and flexible proteins to be determined by cryogenic-electron microscopy (cryo-EM). Recombinant vectors for producing protein double-shell nanostructures are also provided. The nanostructures described herein may find use in various applications in research and drug discovery.

### STATEMENT REGARDING FEDERALLY SPONSORED RESEARCH OR DEVELOPMENT

This invention was made with Government support under contracts A1120943, GM079429, GM103832, and OD021600 awarded by the National Institutes of Health. The Government has certain rights in the invention.

### INCORPORATION BY REFERENCE OF SEQUENCE LISTING PROVIDED AS A TEXT FILE

A Sequence Listing is provided herewith in a text file, STAN-1797WO_S20-404_ST25, created on Sep. 23, 2021 and having a size of 9,546 bytes. The contents of the text file are incorporated herein by reference in its entirety

### BACKGROUND OF THE INVENTION

Recent technological breakthroughs in single-particle cryo-electron microscopy (cryo-EM) have achieved numerous high-resolution structures of macromolecules. For specimens of smaller than 50 kDa that cannot be crystallized or imaged by nuclear magnetic resonance (NMR), cryo-EM is also difficult to be applied, leading to a big gap in the field of structural biology. Extensive efforts have been made to visualize small proteins, including optimization of sample preparation (Herzik et al. Nat. Commun. 10, 1032 (2019)), application of phase plate (Khoshouei et al. Nat. Commun. 8, 16099 (2017); Fan et al. Nat. Commun. 10, 2386 (2019)), and the design of nano-cage systems that link the small proteins to larger molecules with a known structure (Liu et al. Proc. Natl. Acad. Sci. U.S.A. 115, 3362-3367 (2018); Liu et al. Nature Communications 10, 1864 (2019); Coscia et al. Sci. Rep. 6, 30909 (2016); Yao et al. Structure 27, 1148-1155. e3 (2019)). However, the molecular weight of the smallest protein determined by cryo-EM at better than 3.5 Å is still higher than 50 kDa (Fan et al., supra). Besides proteins, some small RNAs less than 50 kDa have been studied by cryo-EM, achieving 3.7 Å for a 40-kDa SAM-IV riboswitch (Zhang et al. Nat. Commun. 10, 5511 (2019)), and 9 Å for a 30-kDa HIV-1 DIS dimer (Zhang et al. Structure 26, 490-498. e3 (2018)), which may be attributed to the high contrast of the phosphate backbone under an electron microscope. However, to date, no polypeptides below 40 kDa have been resolved to better than 4 Å by single particle cryo-EM. Therefore, better methods of determining structures of small proteins are needed.

### SUMMARY OF THE INVENTION

Protein double-shell nanostructures comprising apoferritin for carrying cargo proteins of interest are provided. Such nanostructures can be used to increase rigidity of a cargo protein of interest to allow structures of small and flexible proteins to be determined by cryogenic-electron microscopy (cryo-EM). Recombinant vectors for producing protein double-shell nanostructures are also provided. The nanostructures described herein may find use in various applications in research and drug discovery.

In one aspect, a protein double-shell nanostructure is provided, the nanostructure comprising: a) an inner shell comprising a plurality of apoferritin proteins; b) a cargo protein of interest, wherein the cargo protein is connected to the N-terminus of the apoferritin; and c) an outer shell comprising a tag protein, wherein the tag protein is connected to the cargo protein of interest such that the tag protein points outward from the inner shell and increases rigidity of the cargo protein of interest.

In certain embodiments, the inner shell consists of 24 apoferritin proteins.

In certain embodiments, the cargo protein of interest is smaller than 50 kilodaltons (kDa). In some embodiments, the cargo protein of interest ranges in size from 11 kDa to 50 kDa.

In certain embodiments, the tag protein is a maltose-binding protein (MBP).

In certain embodiments, the apoferritin protein is a truncated apoferritin protein lacking up to the first five N-terminal amino acids residues of SEQ ID NO:1. For example, the apoferritin may have position 1 deleted, positions 1 and 2 deleted, positions 1-3 deleted, positions 1-4 deleted, or positions 1-5 deleted, numbered relative to the reference sequence of SEQ ID NO; 1. In some embodiments, the apoferritin protein is a truncated apoferritin protein consisting of amino acids 6 to 181 numbered relative to the reference sequence of SEQ ID NO:1.

In certain embodiments, the apoferritin comprises a substitution of a cysteine at a position corresponding to D93, E95, and E163 of the apoferritin numbered relative to the reference sequence of SEQ ID NO:1. In some embodiments the apoferritin further comprises a substitution of a serine at a position corresponding to C103 of the apoferritin numbered relative to the reference sequence of SEQ ID NO:1. In some embodiments, the protein double-shell nanostructure further comprises a disulfide bond between a cysteine of the apoferritin and a cysteine of the cargo protein. In some embodiments, cysteine of the apoferritin or the cysteine of the cargo protein is a naturally occurring cysteine or an engineered cysteine mutation.

In certain embodiments, the cargo protein of interest comprises a KIX domain. In some embodiments, the KIX domain is a truncated KIX domain consisting of amino acids 1-80 of the KIX domain numbered relative to the reference sequence of SEQ ID NO:2. In some embodiments, the KIX domain comprises a substitution of a cysteine at a position corresponding to T27 and A31 of the KIX domain numbered relative to the reference sequence of SEQ ID NO:2.

In certain embodiments, the cargo protein retains biological activity within the protein double-shell nanostructure.

In certain embodiments, the protein double-shell nanostructure further comprises a linker. In some embodiments, the protein double-shell nanostructure comprises a linker between the cargo protein and the tag protein and/or a linker between the cargo protein and the apoferritin.

In another aspect, a complex comprising the protein double-shell nanostructure described herein and a binding agent is provided, wherein the binding agent binds to the cargo protein within the protein double-shell nanostructure. In certain embodiments, the binding agent is a substrate, inhibitor, agonist, antagonist, or ligand of the cargo protein.

In another aspect, a method of performing single-particle cryogenic-electron microscopy (cryo-EM) using a protein double-shell nanostructure described herein to determine the structure of the cargo protein of interest is provided, the method comprising: a) providing the protein double-shell nanostructure; and b) performing single-particle cryo-EM on the protein double-shell nanostructure to determine the structure of the cargo protein within the protein double-shell nanostructure.

In certain embodiments, the method further comprises contacting the protein double-shell nanostructure with a binding agent prior to said performing single-particle cryo-EM on the protein double-shell nanostructure, wherein the binding agent binds to the cargo protein within the protein double-shell nanostructure and performing cryo-EM on a complex of the protein double-shell nanostructure with the binding agent.

In certain embodiments, the method further comprises using the cryo-EM structure of the cargo protein to identify a small molecule that binds to the cargo protein, the method comprising: a) screening in silico a small molecule library for candidate small molecules likely to bind to the cargo protein using a three-dimensional model of the cargo protein that is computationally derived from the atomic coordinates of the cryo-EM structure of the cargo protein; and b) evaluating the candidate small molecules identified in step (a) as likely to bind to the cargo protein for their ability to effect activity of the cargo protein using one or more in vitro or in vivo assays to identify at least one candidate small molecule that inhibits or activates activity of the cargo protein.

In another aspect, a fusion protein is provided, the fusion protein comprising: a) an apoferritin protein; b) a cargo protein of interest, wherein the cargo protein is connected to the N-terminus of the apoferritin; and c) a tag protein, wherein the tag protein is connected to the cargo protein of interest.

In another aspect, a vector comprising an expression cassette for expressing a fusion protein described herein is provided.

In certain embodiments, the vector is a non-viral or a viral vector.

In certain embodiments, the expression cassette comprises a promoter operably linked to a coding sequence encoding the fusion protein.

In certain embodiments, the expression cassette comprises: a) a coding sequence encoding the tag protein; b) a coding sequence encoding the apoferritin protein; and c) a multiple cloning site for insertion of a coding sequence encoding the cargo protein of interest in-frame between the coding sequence encoding the tag protein and the coding sequence encoding the apoferritin protein.

In certain embodiments, the expression cassette comprises a multiple cloning site for insertion of a coding sequence encoding the fusion protein.

In another aspect, a method of producing a protein double-shell nanostructure is provided, the method comprising: a) transfecting a host cell with a vector described herein; and b) culturing the host cell under conditions suitable for expression of the fusion protein from the vector, wherein the fusion protein assembles into the protein double-shell nanostructure.

In another aspect, a kit is provided comprising a protein double-shell nanostructure, a fusion, or a vector for producing a fusion protein, as described herein.

### BRIEF DESCRIPTION OF THE DRAWINGS

FIGS. 1A-1D. The design of double-shell system. FIG. 1A. The double shell system is shown by cartoon with apoferritin as inner cage, MBP as outer cage, and small cargo proteins in the middle portion. FIG. 1B. Strategy of plasmid construction. The cargo protein KIX C-terminus is fused to the apoferritin N-terminus with the C-terminal 668-672 residues of KIX and N-terminal 1-5 residues of apoferritin truncated for stability. Several mutations (shown as lightning) were also incorporated on the interface between KIX and apoferritin to enhance stability. The MBP tag is fused to the KIX N-terminus. FIG. 1C. Design of mutations that stabilize the KIX domain of CBP on the apoferritin complex. The diagram of the protein construct with mutations (top). The three-dimensional arrangement of the KIX domain of CBP on the apoferritin complex (bottom). The positions of the five residues that were mutated to cysteine or serine were shown. FIG. 1D. Fluorescence polarization assay using the BODIPY-conjugated phosphorylated KID domain of CREB and KIX, MBP-KIX-apoferritin construct 3, or MBP-apoferritin. KIX, MBP-KIX-apoferritin construct 3, or MBP-apoferritin were titrated.

FIGS. 2A-2F. Single-particle cryo-EM analysis of the MBP-KIX-apoferritin complex (MKF). FIG. 2A. Representative motion-corrected cryo-EM micrograph. FIG. 2B. Reference-free 2D class averages. FIG. 2C. The reconstructed cryo-EM map in two different views. FIG. 2D. Gold standard FSC plot for the final 3D reconstruction, calculated in cryoSPARC. FIG. 2E. Q-score for each amino acid residue in model and (2.5-4) A map of MKF; the orange and grey lines represent the expected Q-scores at 2.5 Å and 4.0 Å, respectively, based on the correlation between Q-scores and map resolution. Right: The model is shown as ribbon, with residue Q-scores indicated. The higher Q-score indicates better resolvability. FIG. 2F. Left, cryo-EM density of two adjacent subunits in two different views. Right, zoom-in view to show the two disulfide bonds between KIX and apoferritin, and the positions of three helices are annotated.

FIGS. 3A-3D. KIX-binding peptide design and the cryo-EM map of peptide bound to MKF. FIG. 3A. List of the designed KIX-binding peptides (left panel). N- and C-termini of the peptides are capped by acetylation and amidation, respectively. Fluorescence polarization assay using the BODIPY-conjugated phosphorylated KID domain of CREB and the KIX domain of CBP with KIX-binding peptides (right panel). The average of fluorescence polarization was plotted with the standard deviation (n=3). FIG. 3B. Left, the reconstructed cryo-EM map of MKF-peptide-7 in two different views. Right, zoom-in view of the extra density after the modeling of KIX domain. FIG. 3C. Cryo-EM density of single KIX-peptide-7 with model fitted. KIX and peptide-7 are depicted as violet red and gold, respectively; the helical-like density corresponding to peptide-7 is separable and shown with gold transparent surface. FIG. 3D. Superimposing models of MKF (grey) and MKF-peptide-7 (green) to demonstrate the KIX domain movement. The peptide-7 in MKF-peptide-7 is hidden for better display.

FIGS. 4A-4F. All designed constructs of the MKF in this study. FIG. 4A. Diagram of four constructs. Rectangles in various colors represent different proteins. FIG. 4B. A potential model of the KIX-apoferritin fusion protein complex. The KIX domain and apoferritin were colored magenta and blue, respectively. T27 and A31 residues in the KIX domain and E169, C177, and E237 residues in apoferritin were colored yellow and cyan, respectively. FIGS. 4C-4F. Cryo-EM 2D class-averages of the four constructs (1-4, respectively).

FIGS. 5A-5B. Workflow of cryo-EM data processing and resolution maps of MKF. FIG. 5A. Workflow of the data processing. FIG. 5B. Resolution maps for the MKF at different thresholds. Upper lane, whole map view; lower lane, slice view.

FIGS. 6A-6C. Single-particle cryo-EM analysis of the MKF-peptide-7 complex. FIG. 6A. Representative motion-corrected cryo-EM micrograph. FIG. 6B. Reference-free 2D class averages.

FIG. 6C. Workflow of the data processing.

FIGS. 7A-7D. Map quality and model validation of MKF-peptide-7. FIG. 7A. Gold standard FSC plot for the final 3D reconstruction, calculated in cryoSPARC. FIG. 7B. Resolution map for the final 3D reconstruction in two different views. FIG. 7C. Left: Q-score for each amino acid residue in model and (2.5-4) A map of MKF in MKF-peptide-7; the orange and grey lines represent the expected Q-scores at 2.5 Å and 4.0 Å, respectively, based on the correlation between Q-scores and map resolution. Right: The model is shown as ribbon, with residue Q-scores indicated. The higher Q-score indicates better resolvability. FIG. 7D. Left: Q-score for each amino acid residue in model and ˜4.5 Å map of peptide-7 in MKF-peptide-7; the orange line represents the expected Q-scores at 4.5 Å. Right: The model is shown as ribbon, with residue Q-scores indicated.

FIGS. 8A-8D. Comparison of previously published NMR data to our cryo-EM structure. FIG. 8A. The cryo-EM structure of the KIX domain (violet) bound to the truncated pKID (gold, PDB ID: 1 KDX). FIG. 8B. The NMR structure of the KIX domain (grey) bound to the truncated pKID (black).

FIG. 8C. NMR structure overlaid with cryo-EM data. FIG. 8A-8C. Three representative views of KIX-KID orientations are shown for the NMR, cryo-EM, and overlaid structures. FIG. 8D. Angle between the truncated pKID (black) and peptide-7 (gold).

### DETAILED DESCRIPTION OF THE INVENTION

Protein double-shell nanostructures comprising apoferritin for carrying cargo proteins of interest are provided. Such nanostructures can be used to increase rigidity of a cargo protein of interest to allow structures of small and flexible proteins to be determined by cryogenic-electron microscopy (cryo-EM). Recombinant vectors for producing protein double-shell nanostructures are also provided. The nanostructures described herein may find use in various applications in research and drug discovery.

Before the present protein double-shell nanostructures and methods of using them are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a protein includes a plurality of such proteins and reference to “the protein” includes reference to one or more proteins and equivalents thereof, e.g. peptides or polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “polypeptide” and “protein” refer to a polymer of amino acid residues and are not limited to a minimum length. Thus, peptides, oligopeptides, dimers, multimers, and the like, are included within the definition. Both full length proteins and fragments thereof are encompassed by the definition. The terms also include post-expression modifications of the polypeptide, for example, glycosylation, acetylation, phosphorylation, hydroxylation, and the like. Furthermore, for purposes of the present invention, a “polypeptide” refers to a protein which includes modifications, such as deletions, additions and substitutions to the native sequence, so long as the protein maintains the desired activity. These modifications may be deliberate, as through site directed mutagenesis, or may be accidental, such as through mutations of hosts which produce the proteins or errors due to PCR amplification.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

Substantially purified” generally refers to isolation of a substance (compound, nanostructure, fusion protein, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The terms “fusion protein” or “fusion polypeptide,” as used herein refer to a fusion comprising apoferritin in combination with a cargo protein of interest and a tag protein as part of a single continuous chain of amino acids, which chain does not occur in nature. The fusion protein may also contain additional sequences, such as targeting or localization sequences, detectable labels, or tag sequences.

The term “apoferritin” as used herein encompasses all forms of apoferritin and also includes biologically active fragments, variants, analogs, and derivatives thereof that retain the ability to form the inner shell of a protein double-shell nanostructure and increase the rigidity of a cargo protein of interest, as described herein.

An apoferritin polynucleotide, nucleic acid, oligonucleotide, protein, polypeptide, or peptide refers to a molecule derived from any source. The molecule need not be physically derived from an organism, but may be synthetically or recombinantly produced. A number of apoferritin nucleic acid and protein sequences are known. A representative sequence of murine apoferritin is presented in SEQ ID NO:1. Additional representative sequences, including sequences from other species are listed in the National Center for Biotechnology Information (NCBI) database. See, for example, NCBI entries: Accession Nos. NP_002023, CAA27205, NP_001238983, NP_002023, NP_034369, AAA37612, 7KOD_X, NP_776487, NP_036980, XP_001060160, EHH64025, XP_015289650, XP_032976293, KAF6333100, XP_030416668, NP_990417, NP_001009786, NP_001180585, NP_001003080, NP 999140, NP_00104161, and NP_001166318; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce a fusion protein or recombinant polynucleotide comprising a coding sequence encoding a apoferritin protein for use in construction of a protein double-shell nanostructure, as described herein.

By “fragment” is intended a molecule consisting of only a part of the intact full-length sequence and structure. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, DC, which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells. The term refers to both stable and transient uptake of the genetic material, and includes uptake, for example, of recombinant nucleic acids encoding fusion proteins.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Operably linked” refers to an arrangement of elements wherein the components so described are configured so as to perform their usual function. For example, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

The terms “variant” refers to biologically active derivatives of the reference molecule that retain desired activity. In general, the term “variant” refers to molecules having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

The terms “subject” refers to a vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; and birds, including domestic, wild and game birds such as chickens, turkeys and other gallinaceous birds, ducks, geese, and the like. The term does not denote a particular age. Thus, both adult and newborn individuals are intended to be covered.

### Fusion Proteins and Assembly of Protein Double-Shell Nanostructures

Fusion proteins comprising an inner shell apoferritin protein, a cargo protein of interest, and an outer shell tag protein can assemble into protein double-shell nanostructures as described herein (see Example 1). In some embodiments, the cargo protein is connected to the N-terminus of the apoferritin and the tag protein is connected to the C-terminus of the cargo protein. In the fusion protein, the cargo protein may be connected directly to the apoferritin and the tag protein by peptide bonds or may be separated by intervening amino acid sequences (i.e., linker). The fusion protein may also contain sequences exogenous to the apoferritin, cargo protein, and tag protein. For example, the fusion may include targeting or localization sequences, detectable labels, or additional tag sequences.

In certain embodiments, the fusion protein can be represented by the formula NH₂-A-tag protein-L-cargo protein-L-apoferritin-B—COOH, wherein: L is an optional linker amino acid sequence; A is an optional N-terminal amino acid sequence; and B is an optional C-terminal amino acid sequence.

Linker amino acid sequence(s) -L- will typically be short, e.g., 20 or fewer amino acids (i.e., 20, 19, 18, 17, 16, 15, 14, 13, 12, 11, 10, 9, 8, 7, 6, 5, 4, 3, 2, or 1). Examples include short peptide sequences which facilitate cloning, poly-glycine linkers (Glyₙ where n=2, 3, 4, 5, 6, 7, 8, 9, 10 or more), histidine tags (Hisₙ where n=3, 4, 5, 6, 7, 8, 9, 10 or more), linkers composed of glycine and serine residues, GSAT, SEG, and Z-EGFR linkers. Linkers may include restriction sites, which aid cloning and manipulation. Other suitable linker amino acid sequences will be apparent to those skilled in the art. (See e.g., Argos (1990) J. Mol. Biol. 211(4):943-958; Crasto et al. (2000) Protein Eng. 13:309-312; George et al. (2002) Protein Eng. 15:871-879; Arai et al. (2001) Protein Eng. 14:529-532; and the Registry of Standard Biological Parts (partsregistry.org/Protein_domains/Linker).

-A- is an optional N-terminal amino acid sequence. This will typically be short, e.g., 40 or fewer amino acids (i.e., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). Examples include leader sequences to direct protein localization, or short peptide sequences or tag sequences, which facilitate cloning or purification (e.g., a histidine tag Hisₙ where n=3, 4, 5, 6, 7, 8, 9, 10 or more). Other suitable N-terminal amino acid sequences will be apparent to those skilled in the art.

-B- is an optional C-terminal amino acid sequence. This will typically be short, e.g., 40 or fewer amino acids (i.e., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). Examples include sequences to direct protein localization, short peptide sequences or tag sequences, which facilitate cloning or purification (e.g., Hisₙ where n=3, 4, 5, 6, 7, 8, 9, 10 or more), or sequences which enhance protein stability. Other suitable C-terminal amino acid sequences will be apparent to those skilled in the art.

In certain embodiments, tag sequences are located at the N-terminus of the fusion protein. Exemplary tags that can be used in the practice of the invention include a maltose-binding protein tag, a His-tag, a Strep-tag, a TAP-tag, an S-tag, an SBP-tag, an Arg-tag, a calmodulin-binding peptide tag, a cellulose-binding domain tag, a DsbA tag, a c-myc tag, a glutathione S-transferase tag, a FLAG tag, a HAT-tag, a NusA tag, and a thioredoxin tag.

Apoferritin nucleic acid and protein sequences may be derived from any source. A number of apoferritin nucleic acid and protein sequences are known. A representative sequence of murine apoferritin is presented in SEQ ID NO:1. Additional representative sequences, including sequences from other species are listed in the National Center for Biotechnology Information (NCBI) database. See, for example, NCBI entries: Accession Nos. NP_002023, CAA27205, NP_001238983, NP_002023, NP_034369, AAA37612, 7KOD_X, NP_776487, NP_036980, XP_001060160, EHH64025, XP_015289650, XP_032976293, KAF6333100, XP_030416668, NP_990417, NP_001009786, NP_001180585, NP_001003080, NP_999140, NP_00104161, and NP_001166318; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce a fusion protein or recombinant polynucleotide comprising a coding sequence encoding an apoferritin protein for use in construction of a protein double-shell nanostructure, as described herein.

In certain embodiments, the apoferritin protein is truncated to remove flexible regions. In some embodiments, the fusion protein comprises an apoferritin protein lacking up to the first five N-terminal amino acids residues of SEQ ID NO:1. For example, the apoferritin may have position 1 deleted, positions 1 and 2 deleted, positions 1-3 deleted, positions 1-4 deleted, or positions 1-5 deleted at the N-terminus. In some embodiments, the apoferritin protein is a truncated apoferritin protein consisting of amino acids 6 to 181 of the apoferritin numbered relative to the reference sequence of SEQ ID NO:1. In certain embodiments, cysteine residues are introduced into the apoferritin and/or the cargo protein to generate disulfide bonds in order to increase rigidity of the molecules in the protein double-shell nanostructure. In certain embodiments, the apoferritin comprises a substitution of a cysteine at a position corresponding to D93, E95, and E163 of the apoferritin numbered relative to the reference sequence of SEQ ID NO:1. In some embodiments the apoferritin further comprises a substitution of a serine at a position corresponding to C103 of the apoferritin numbered relative to the reference sequence of SEQ ID NO:1. In some embodiments, the protein double-shell nanostructure further comprises a disulfide bond between a cysteine of the apoferritin and a cysteine of the cargo protein. In some embodiments, the cysteine of the apoferritin or the cysteine of the cargo protein is a naturally occurring cysteine or an engineered cysteine mutation. The foregoing numbering is relative to murine apoferritin (SEQ ID NO:1), but it is to be understood that the corresponding positions in apoferritin obtained from other species are also intended to be encompassed by the present invention.

Fusion proteins can be prepared in any suitable manner (e.g., recombinant expression, purification from cell culture, chemical synthesis, etc.). Fusion proteins may include naturally occurring polypeptides, recombinantly produced polypeptides, synthetically produced polypeptides, or polypeptides produced by a combination of these methods. Means for preparing fusion proteins are well understood in the art. Fusion proteins are preferably prepared in substantially pure form (i.e. substantially free from other host cell or non-host cell proteins).

In one embodiment, the fusion proteins are generated using recombinant techniques. One of skill in the art can readily determine nucleotide sequences that encode the desired polypeptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the claimed fusion proteins that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage A (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), Ylp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA (“MaxBac” kit).

Plant expression systems can also be used to produce the fusion proteins described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia-based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases, it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fusion proteins of the present invention are produced by growing host cells transformed by an expression vector described above under conditions whereby the fusion protein of interest is expressed. The selection of the appropriate growth conditions is within the skill of the art.

In one embodiment, the transformed cells secrete the fusion protein product into the surrounding media. Certain regulatory sequences can be included in the vector to enhance secretion of the protein product, for example using a tissue plasminogen activator (TPA) leader sequence, an interferon (y or a) signal sequence or other signal peptide sequences from known secretory proteins. The secreted fusion protein product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant fusion proteins substantially intact. Intracellular proteins can also be obtained by removing components from the cell wall or membrane, e.g., by the use of detergents or organic solvents, such that leakage of the proteins occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced fusion proteins are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular fusion proteins of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the fusion proteins can be further purified using conventional techniques well known in the art, such as by any of the techniques described above.

Fusion proteins can also be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final peptide or polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, I L 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides, including fusion proteins.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

Fusion proteins can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

The fusion proteins self-assemble into multimeric complexes to produce protein double-shell nanostructures (see Examples). In some embodiments, the fusion proteins assemble into a protein double-shell nanostructure consisting of a 24-mer fusion protein complex. Protein double-shell nanostructures can be purified from mixtures containing contaminants such as fusion protein monomers and partially assembled nanostructures using, for example, high resolution gel filtration, electrophoresis, differential or density gradient centrifugation, or reverse phase or ion exchange chromatography. In some embodiments, the protein double-shell nanostructure is substantially purified to at least 50%, preferably at least 80%-85%, or more preferably at least 90-95% purity.

Cryogenic-Electron Microscopy and Screening

Methods of performing cryogenic-electron microscopy (cryo-EM) on a protein double-shell nanostructure to determine the structure of a cargo protein of interest within the protein double-shell nanostructure are also provided. Protein nanostructures can be used to increase rigidity of a cargo protein of interest to allow structures of small and flexible proteins of 50 kilodaltons or less to be determined by cryo-EM. The apoferritin in the nanostructure acts as an inner cage having high rigidity and symmetry, which facilitates determination of atomic resolution cryo-EM structures, wherein the cargo protein of interest is displayed in the nanostructure rigidly enough to allow the structure of the cargo protein to be determined at atomic resolution without blurring.

In some embodiments, cryo-EM is performed on a protein double-shell nanostructure, wherein the cargo protein of interest ranges in size from 11 kDa to 50 kDa, including any size within this range such as 11 kDa, 12 kDa, 13 kDa, 14 kDa, 15 kDa, 16 kDa, 17 kDa, 18 kDa, 19 kDa, 20 kDa, 22 kDa, 24 kDa, 26 kDa, 28 kDa, 30 kDa, 32 kDa, 34 kDa, 36 kDa, 38 kDa, 40 kDa, 42 kDa, 44 kDa, 46 kDa, 48 kDa, or 50 kDa. In some embodiments, the cargo protein is 50 kDa or less, 45 kDa or less, 40 kDa or less, 35 kDa or less, 30 kDa or less, 25 kDa or less, 20 kDa or less, or 15 kDa or less. In some embodiments, the cargo protein is 50 kDa or more, 75 kDa or more, 100 kDa or more, 200 kDa or more, 300 kDa or more, 400 kDa or more, 500 kDa or more, 600 kDa or more, 700 kDa or more, 800 kDa or more, 900 kDa or more, or 1000 kDa or more.

In some embodiments, electron microscopy is performed on a sample comprising a protein double-shell nanostructure at cryogenic temperatures. Samples are typically cooled to cryogenic temperatures using a plunge freezing technique, in which the sample is applied to a grid-mesh and plunged, for example, into liquid ethane, a mixture of liquid ethane and propane, liquid nitrogen, or liquid helium. In some embodiments, single particle analysis cryo-EM is performed on a protein double-shell nanostructure, which further involves computationally combining images of many individual protein double-shell nanostructures of the same type randomly oriented within a thin layer of vitreous ice. A sufficient number of images of the nanostructure should be used to achieve a suitable signal-to-noise ratio and provide enough different views to produce a three-dimensional structural model. Single particle cryo-EM offers the advantage over X-ray crystallography of producing atomic resolution structures without the need to crystallize a protein. For a description of single-particle cryo-electron microscopy techniques, see, e.g., Cheng et al. (2018) Science 361 (6405):876-880; Cheng et al. (2015) Cell 161:438-449; Fernandez-Leiro et al. (2016) Nature 537:339-346; Nwanochie et al. (2019) Int J. Mol. Sci. 20(17):4186; Danev et al. (2019) Trends Biochem. Sci. 44(10):837-848; Akbar et al. (2020) J. Chem. Inf. Model 60(5):2448-2457; Frank (2017) Nat. Protoc. 12(2):209-212; U.S. Pat. No. 10,559,449; and U.S. Patent Application Publication No. 2020/0167913; herein incorporated by reference.

In some embodiments, cryo-EM is performed on a complex comprising the protein double-shell nanostructure and a binding agent, wherein the binding agent binds to the cargo protein within the protein double-shell nanostructure. The binding agent can be, for example, a substrate, inhibitor, agonist, antagonist, ligand, or drug that binds to the cargo protein. In some embodiments, the cargo protein within the protein double-shell nanostructure is contacted with the binding agent before performing cryo-EM to obtain a structure of a complex of the protein double-shell nanostructure with the binding agent bound to the cargo protein of interest.

The structures of cargo proteins (in the presence or absence of binding agents), produced by single particle cryo-EM using protein double-shell nanostructures, can be used in various applications in basic research and drug development. In particular, cryo-EM structures of cargo proteins can be used in rational drug design to identify small molecules that bind to a cargo protein. A small molecule library can be screened in silico for candidate small molecules likely to bind to the cargo protein using a three-dimensional model of the cargo protein that is computationally derived from the atomic coordinates of the cryo-EM structure of the cargo protein. Candidate small molecules, identified as likely to bind to the cargo protein, can then be evaluated for their effects (e.g., inhibition or activation) on the activity of the cargo protein using one or more in vitro or in vivo assays to identify at least one candidate small molecule that inhibits or activates activity of the cargo protein.

For purposes of the assay methods, the cargo protein of interest may be provided as an isolated protein. Alternatively, the cargo protein can be present in the context of a nanostructure. Any convenient format may be used for the assay, e.g. wells, plates, flasks, etc., preferably a high throughput format, such as multi-well plates. A test agent of interest is added to the reaction mixture with the cargo protein, for example, and the effect of the agent on the activity of the cargo protein is determined.

Inhibitors or activators can be identified by contacting the cargo protein with a candidate agent; and measuring inhibition or activation of the biological activity of the cargo protein by the candidate agent. Assays may further include suitable controls (e.g., a sample comprising the cargo protein in the absence of the test agent). Generally, a plurality of assay mixtures is run in parallel with different agent concentrations to obtain a differential response to the various concentrations. Typically, one of these concentrations serves as a negative control, i.e., at zero concentration or below the level of detection.

A variety of other reagents may be included in the screening assay. These include reagents like salts, neutral proteins, e.g., albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity but may also be optimized to facilitate rapid high-throughput screening. In some embodiments, between 0.1 hour and 1 hour, between 1 hour and 2 hours, or between 2 hours and 4 hours, will be sufficient.

A variety of different test agents may be screened. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, proteins, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2 H-tetrazolium bromide) assay, and the like. Agents that do not exhibit significant cytotoxic activity are considered candidate agents.

Kits

Any of the compositions described herein may be included in a kit. The subject kits may include a protein double-shell nanostructure comprising a cargo protein of interest, fusion proteins for assembling such a protein double-shell nanostructure, as described herein, or vectors encoding such fusion proteins.

Compositions can be in liquid form or can be lyophilized, as can individual protein double-shell nanostructures, fusion proteins, or vectors. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A protein double-shell nanostructure comprising:
   a) an inner shell comprising a plurality of apoferritin proteins;
   b) a cargo protein of interest, wherein the cargo protein is connected to the N-terminus of the apoferritin; and
   c) an outer shell comprising a tag protein, wherein the tag protein is connected to the cargo protein of interest such that the tag protein points outward from the inner shell and increases rigidity of the cargo protein of interest.
2. The protein double-shell nanostructure of aspect 1, wherein the inner shell consists of 24 apoferritin proteins.
3. The protein double-shell nanostructure of aspect 1 or 2, wherein the cargo protein of interest is smaller than 50 kilodaltons (kDa).
4. The protein double-shell nanostructure of aspect 3, wherein the cargo protein of interest ranges in size from 11 kDa to 50 kDa.
5. The protein double-shell nanostructure of any one of aspects 1-4, wherein the tag protein is a maltose-binding protein (MBP).
6. The protein double-shell nanostructure of any one of aspects 1-5, wherein the apoferritin protein is a truncated apoferritin protein lacking up to the first five N-terminal amino acids residues of SEQ ID NO:1.
7. The protein double-shell nanostructure of any one of aspects 1-6, wherein the apoferritin protein is a truncated apoferritin protein consisting of amino acids 6 to181 of apoferritin numbered relative to the reference sequence of SEQ ID NO:1.
8. The protein double-shell nanostructure of any one of aspects 1-7, wherein the apoferritin comprises a substitution of a cysteine at a position corresponding to D93, E95, and E163 of the apoferritin numbered relative to the reference sequence of SEQ ID NO:1.
9. The protein double-shell nanostructure of aspect 8, wherein the apoferritin further comprises a substitution of a serine at a position corresponding to C103 of the apoferritin numbered relative to the reference sequence of SEQ ID NO:1.
10. The protein double-shell nanostructure of aspect 8 or 9, further comprising a disulfide bond between a cysteine of the apoferritin and a cysteine of the cargo protein.
11. The protein double-shell nanostructure of aspect 10, wherein the cysteine of the apoferritin or the cysteine of the cargo protein is a naturally occurring cysteine or an engineered cysteine mutation.
12. The protein double-shell nanostructure of any one of aspects 1-11, wherein the cargo protein of interest comprises a KIX domain.
13. The protein double-shell nanostructure of aspect 12, wherein the KIX domain is a truncated KIX domain consisting of amino acids 1-80 of the KIX domain numbered relative to the reference sequence of SEQ ID NO:2.
14. The protein double-shell nanostructure of aspect 12 or 13, wherein the KIX domain comprises a substitution of a cysteine at a position corresponding to T27 and A31 of the KIX domain numbered relative to the reference sequence of SEQ ID NO:2.
15. The protein double-shell nanostructure of any one of aspects 1-14, wherein the cargo protein retains biological activity within the protein double-shell nanostructure.
16. The protein double-shell nanostructure of any one of aspects 1-15, further comprising a linker.
17. The protein double-shell nanostructure of aspect 16, wherein the linker is between the cargo protein and the tag protein.
18. The protein double-shell nanostructure of aspect 16 or 17, wherein the linker is between the cargo protein and the apoferritin.
19. A complex comprising the protein double-shell nanostructure of any one of aspects 1-18 and a binding agent, wherein the binding agent binds to the cargo protein within the protein double-shell nanostructure.
20. The complex of aspect 19, wherein the binding agent is a substrate, inhibitor, agonist, antagonist, or ligand of the cargo protein.
21. A method of performing single-particle cryogenic-electron microscopy (cryo-EM) using the protein double-shell nanostructure of any one of aspects 1-18 to determine the structure of the cargo protein of interest, the method comprising:
   a) providing the protein double-shell nanostructure of any one of aspects 1-18; and
   b) performing single-particle cryo-EM on the protein double-shell nanostructure to determine the structure of the cargo protein within the protein double-shell nanostructure.
22. The method of aspect 21, further comprising contacting the protein double-shell nanostructure with a binding agent prior to said performing single-particle cryo-EM on the protein double-shell nanostructure, wherein the binding agent binds to the cargo protein within the protein double-shell nanostructure, wherein said performing single-particle cryo-EM comprises performing cryo-EM on a complex of the protein double-shell nanostructure with the binding agent.
23. The method of aspect 21 or 22, further comprising using the cryo-EM structure of the cargo protein to identify a small molecule that binds to the cargo protein, the method comprising: a) screening in silico a small molecule library for candidate small molecules likely to bind to the cargo protein using a three-dimensional model of the cargo protein that is computationally derived from the atomic coordinates of the cryo-EM structure of the cargo protein; and b) evaluating the candidate small molecules identified in step (a) as likely to bind to the cargo protein for their ability to effect activity of the cargo protein using one or more in vitro or in vivo assays to identify at least one candidate small molecule that inhibits or activates activity of the cargo protein.
24. A fusion protein comprising:
   a) an apoferritin protein;
   b) a cargo protein of interest, wherein the cargo protein is connected to the N-terminus of the apoferritin; and
   c) a tag protein, wherein the tag protein is connected to the cargo protein of interest.
25. A vector comprising an expression cassette for expressing the fusion protein of aspect 24.
26. The vector of aspect 25, wherein the vector is a non-viral or a viral vector.
27. The vector of aspect 25 or 26, wherein the expression cassette comprises a promoter operably linked to a coding sequence encoding the fusion protein.
28. The vector of aspect 25 or 26, wherein the expression cassette comprises:
   a) a coding sequence encoding the tag protein;
   b) a coding sequence encoding the apoferritin protein; and
   c) a multiple cloning site for insertion of a coding sequence encoding the cargo protein of interest in-frame between the coding sequence encoding the tag protein and the coding sequence encoding the apoferritin protein.
29. The vector of aspect 25 or 26, wherein the expression cassette comprises a multiple cloning site for insertion of a coding sequence encoding the fusion protein.
30. A method of producing a protein double-shell nanostructure, the method comprising:
   a) transfecting a host cell with the vector of any one of aspects 25-29; and
   b) culturing the host cell under conditions suitable for expression of the fusion protein from the vector, wherein the fusion protein assembles into the protein double-shell nanostructure.
31. A kit comprising the protein double-shell nanostructure of any one of aspects 1-18.
32. A kit comprising the fusion protein of aspect 24.
33. A kit comprising the vector of any one of aspects 25-29.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Achieving Near-Atomic Resolution Cryo-EM Structure of an 11-kDa Flexible Protein to Guide Drug Discovery

### INTRODUCTION

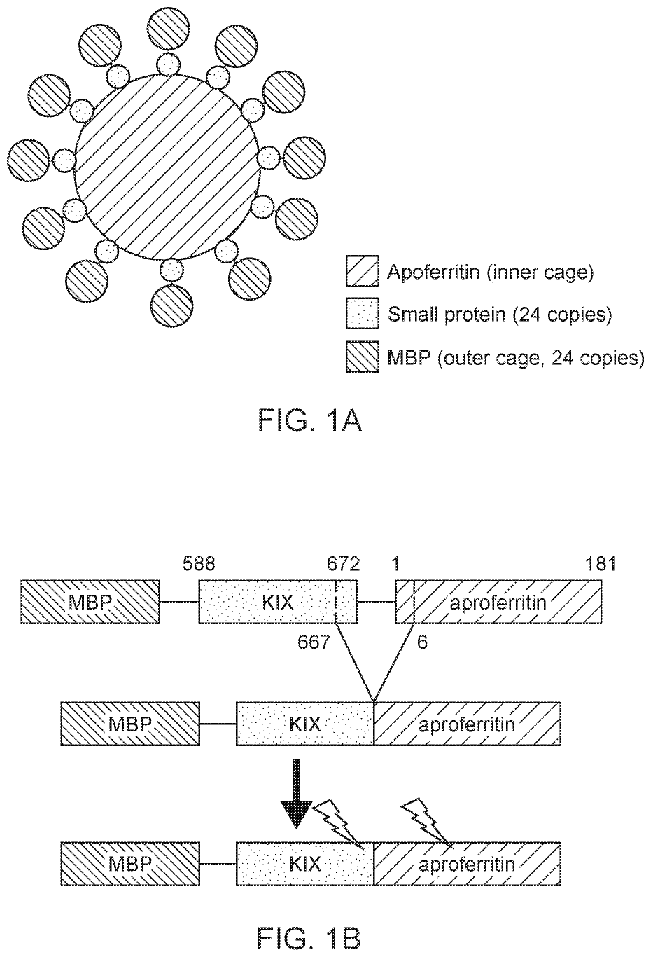

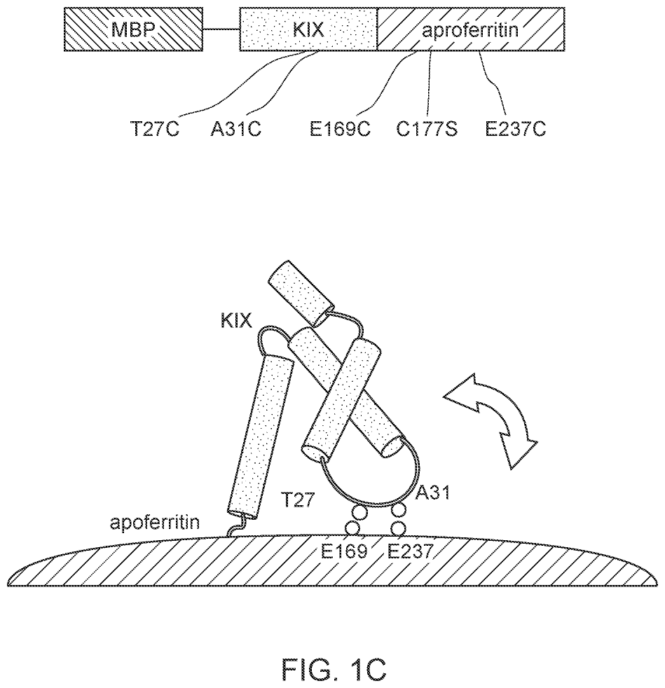

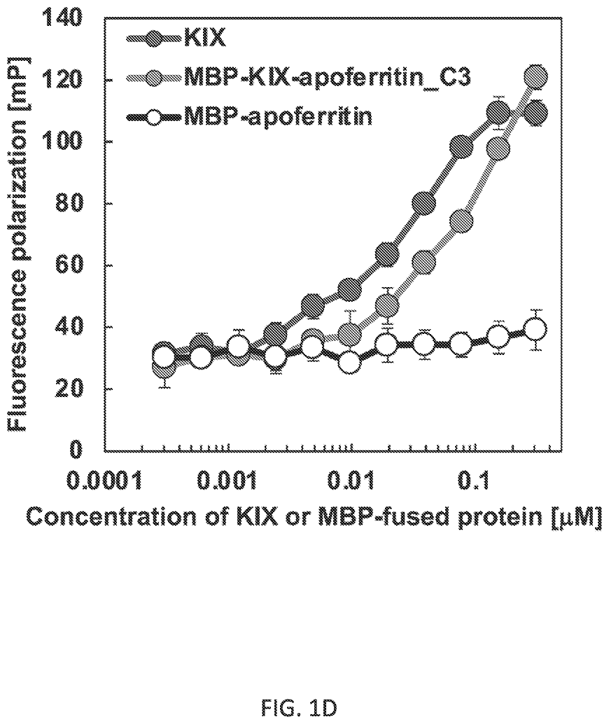

The use of nano-cages in cryo-EM is limited by rigidity and modularity. The cargo protein should be displayed rigidly to avoid being blurred during the reconstruction of the whole complex. Besides, the cage system should be universally applicable to various cargo proteins without requiring big changes. Here, we established a double-shell system that meets the above requirements and can be applied to protein molecules far smaller than 50 kDa. The double-shell system uses the apoferritin as the inner cage owing to its high rigidity, symmetry, and easily achievable atomic resolution cryo-EM structure¹⁰⁻¹². The KIX domain (11 kDa) of CREB-binding protein (CBP) is genetically engineered to fuse directly to the N-terminus of apoferritin, and the maltose-binding protein (MBP) is fused to the KIX domain, thus pointing outwards as an outer shell to protect and rigidify the small protein of interest (FIG. 1A). The KIX domain was selected as the small cargo protein as it has been considered a potent drug target against cancer because of its interaction with the pKID domain of Cyclic AMP Response Element Binding protein (CREB) associated with poor prognosis in acute myeloid leukemia (AML)¹³.

The CBP KIX domain is known to be quite flexible defying high-resolution X-ray structural analysis. Indeed, for a long time, the only known structures of the KIX domain were determined by NMR, until the work by Wang and colleagues¹⁴⁻¹⁵ who conjugated a small compound to stabilize the KIX helices. Unfortunately, the proposed pKID domain-binding site of the KIX is covered by a neighboring KIX molecule in the crystal, making it impossible to do small compound soaking experiments, followed by the development of inhibitors for the CREB-CBP interaction. The cognizant protein domain, pKID, is even more challenging since it is intrinsically disordered. These issues have hindered structure-based protein-protein interaction inhibitor design. In contrast, the cryo-EM single-particle analysis does not rely on the availability of high-quality crystals and there is no need to worry about the crystal contacts which might force proteins in certain conformational states. However, the molecular weight of the KIX domain is only ˜11 kDa, way smaller than the current threshold for high-resolution protein structure determination using cryo-EM, hence a successful structure determination of the KIX domain itself and in complex with its inhibitors using the double-shell scaffolds will verify the practical applicability of single-particle cryo-EM for such small proteins from a technical point of view.

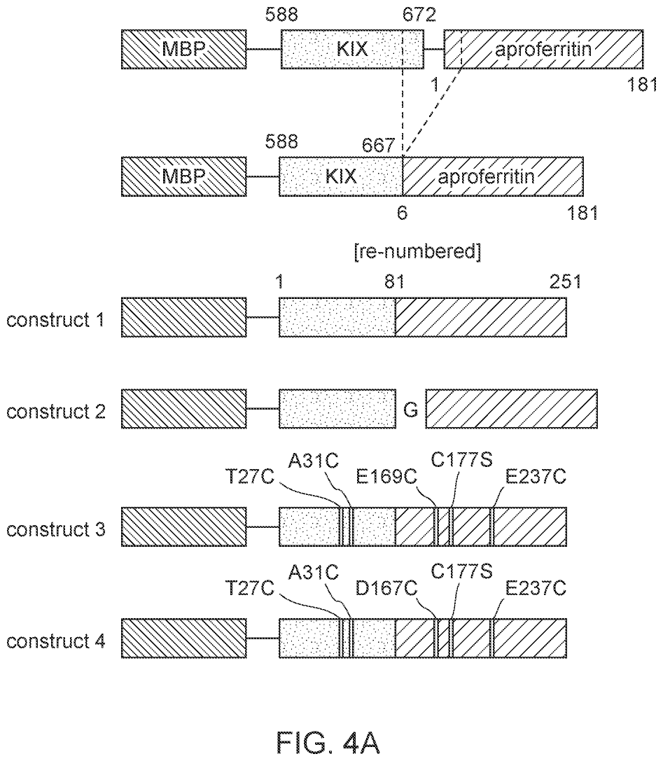

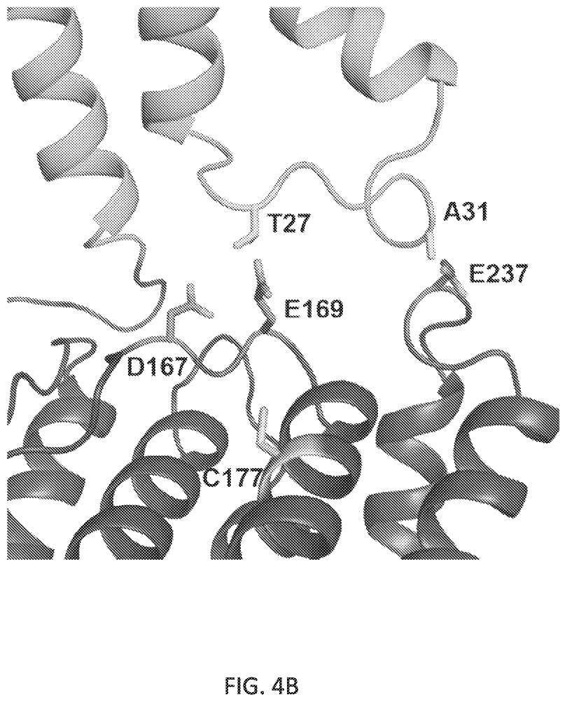

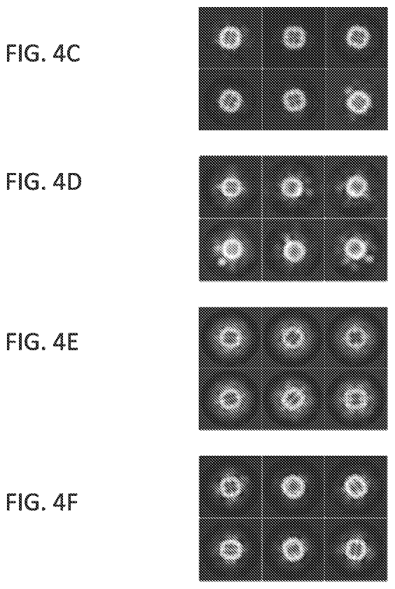

Initially, a chimeric protein was constructed by fusing the KIX domain to apoferritin, in which the C-terminus (residues: 668-672) of the KIX domain and the N-terminus (residues: 1-5) of apoferritin were truncated to reduce possible flexibility (FIG. 1B and FIG. 4A). Here, we renumbered the residues of the KIX domain (588-667) and apoferritin (6-181) to 1-80 (KIX) and 81-251 (apoferritin) (FIG. 4). In all four constructs used for cryo-EM work in this study, an MBP tag was fused with KIX and apoferritin to facilitate protein purification and possibly reduce the air-water interface-induced damage to the KIX domain and apoferritin cage during the specimen vitrification step (FIG. 1B). The initial cryo-EM two-dimensional (2D) analysis of construct 1 and construct 2 of the MBP-KIX-apoferritin revealed that the KIX domain on apoferritin is flexible according to the lacking or missing density outside apoferritin (FIGS. 4C-4D). To improve this issue, we designed cysteine mutations on the KIX domain and apoferritin to stabilize the KIX domain on apoferritin with disulfide bonds (FIG. 1C). By investigating the range of possible orientations of the KIX domain on the apoferritin shell scaffold, we identified possible residue pairs which can form disulfide bonds (FIG. 1c and FIG. 4B). In this second design phase, one KIX molecule crosslinks with one adjacent apoferritin subunit through two disulfide bonds. The renumbered T27 and A31 residues of the KIX domain and E169/D167 and E237 residues of the apoferritin were mutated to cysteine. In addition, C177 was mutated to serine to prevent the formation of unexpected disulfide bonds. We also deleted a glycine residue between KIX and apoferritin in construct 2 due to its induced subtle flexibility, as demonstrated by worse 2D class averages compared with construct 1, which might interrupt disulfide bonding among T27: E169/D167 and A31: E237. Therefore, we designed another two constructs of the MBP-KIX-apoferritin (FIGS. 4A-4B). The specimens were then screened by cryo-EM 2D analysis and the protein specimen derived from construct 3 with the best cryo-EM density in the KIX domain was selected for further data collection and processing (FIGS. 4E-4F). Hereinafter, we refer to this protein from construct 3 of the MBP-KIX-apoFerritin complex as MKF. The MKF complex was also examined for the binding affinity of KIX for the BODIPY-conjugated pKID domain of CREB protein using the fluorescence polarization (FP) assay¹⁶. The results showed that the KIX domains of the 24-mer complex still maintained its function when fused to the double-shell system (FIG. 1D).

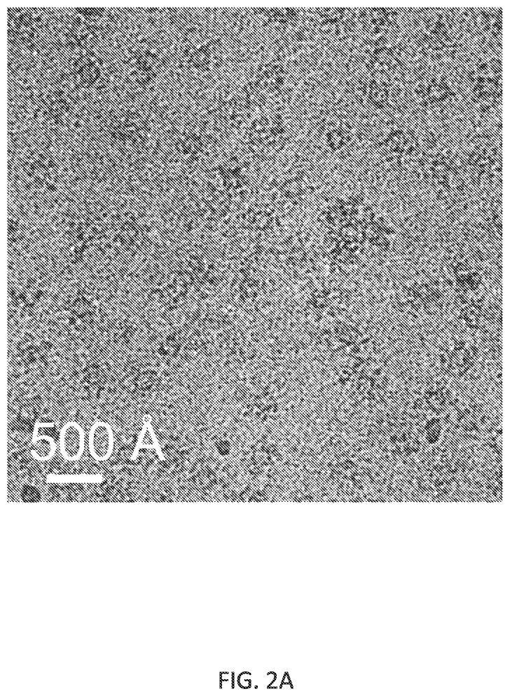

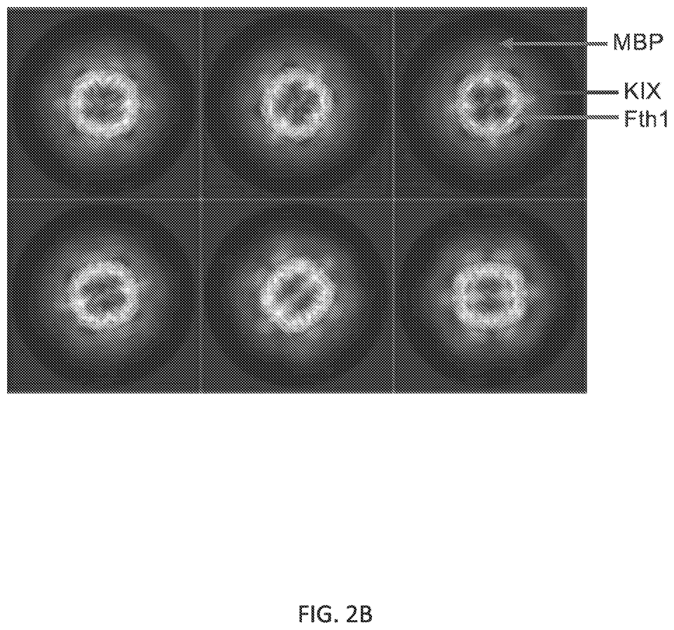

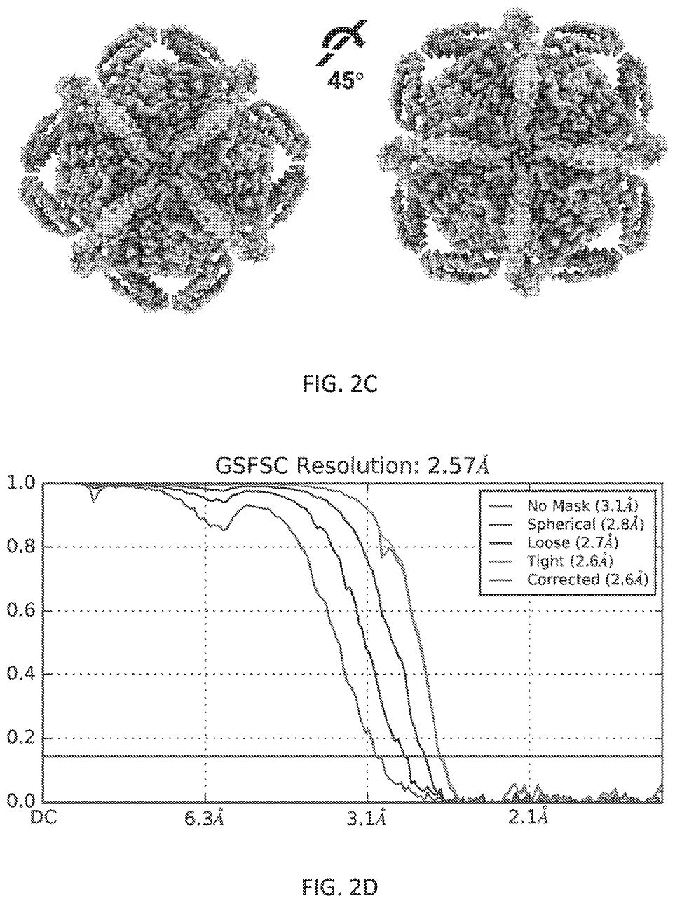

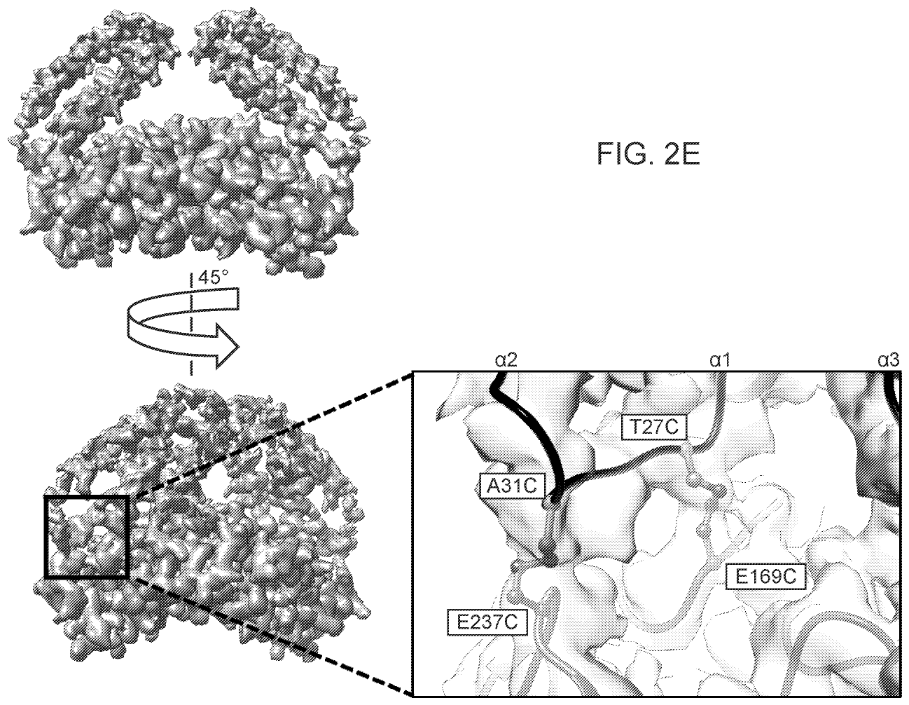

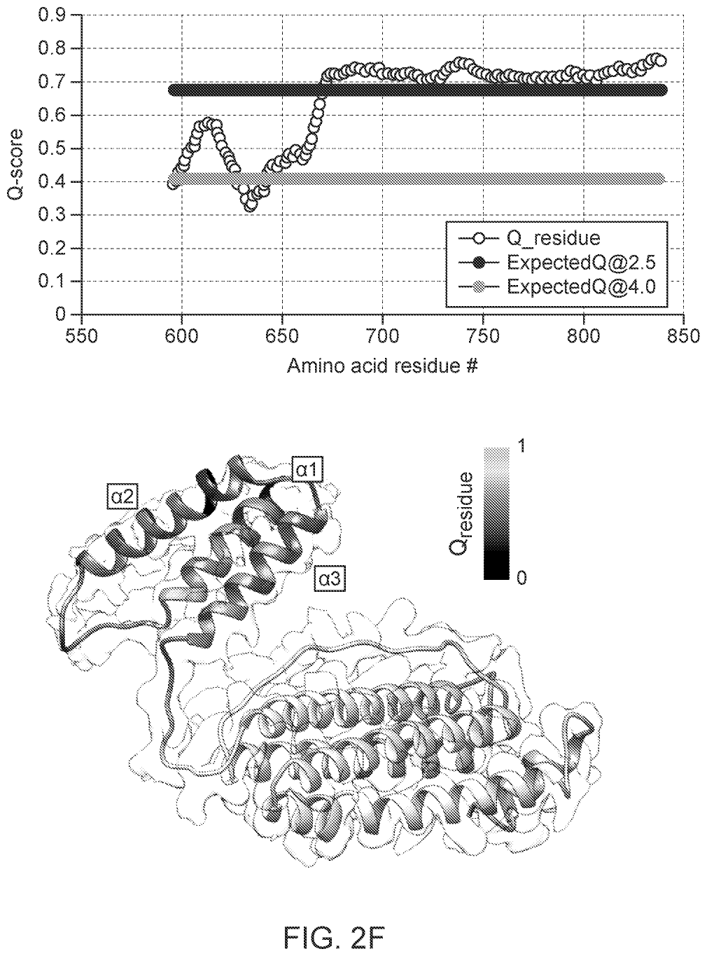

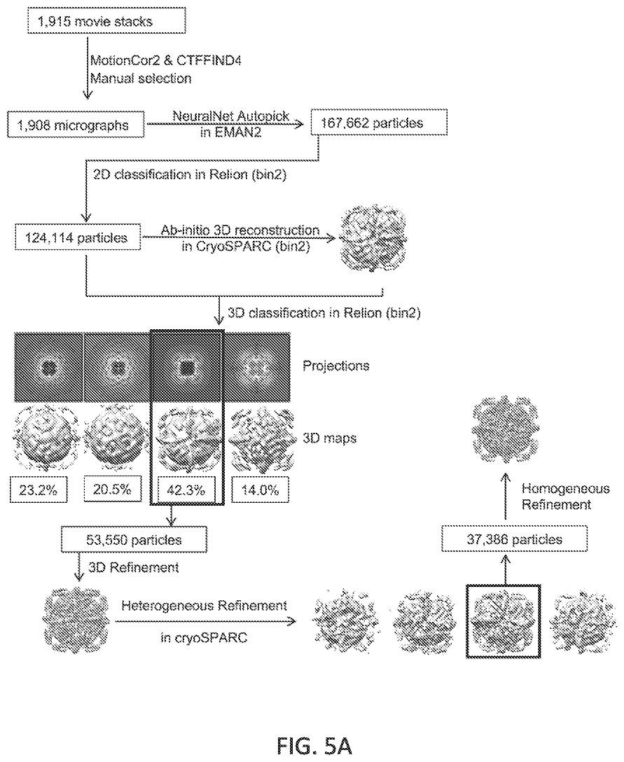

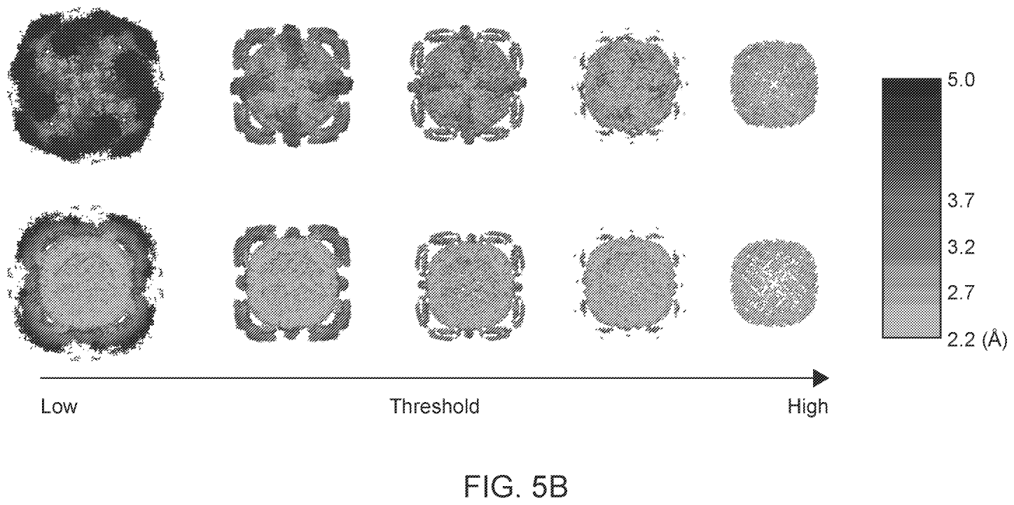

The cryo-EM raw image shows extra densities around apoferritin, indicating the existence of the MKF particle (FIG. 2A). Further 2D class averages clearly display three layers, a smeared outer shell MBP, middle cargo protein KIX, and inner shell apoferritin, which is consistent with our design (FIG. 1 and FIG. 2B). Using single-particle analysis, we determined the three-dimensional (3D) structure of MKF from ˜37,000 particles and achieved an overall resolution of 2.6 Å (FIGS. 2C and 2D, FIG. 5A and Table S1). The apoferritin and KIX domain are well resolved at 2.2-2.5 and 3.0-4.0 Å, respectively, (FIG. 5B), confirmed by Q-scores developed for evaluating the density resolvability (FIG. 2E). However, the MBP portion cannot be resolved presumably due to the high flexibility of the linker between the MBP and KIX domain. Notably, the KIX domain shows a directional resolution distribution; the closer to the apoferritin inner shell, the higher resolution obtained, demonstrating that the KIX domain closer to apoferritin is more stable. The cysteine mutations on T27 and A31 of KIX distributed between two alpha-helix domains form disulfide bonds with the cysteine mutations on E169 and E237 of adjacent apoferritin, respectively, may be attributable to the increased stability of the KIX structure (FIG. 2F).

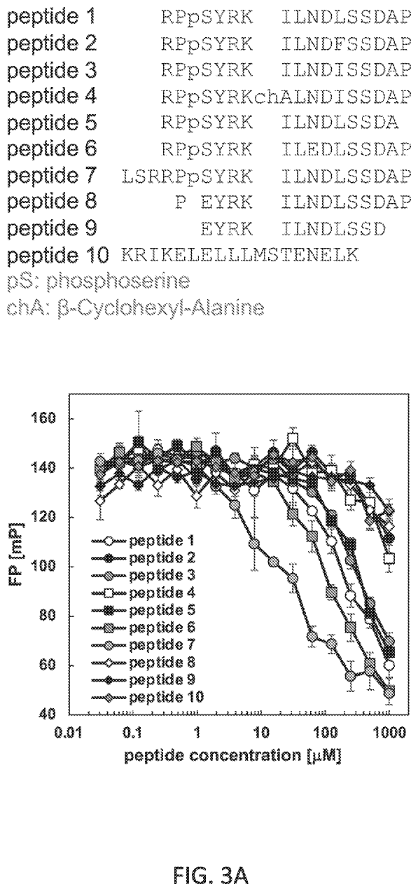

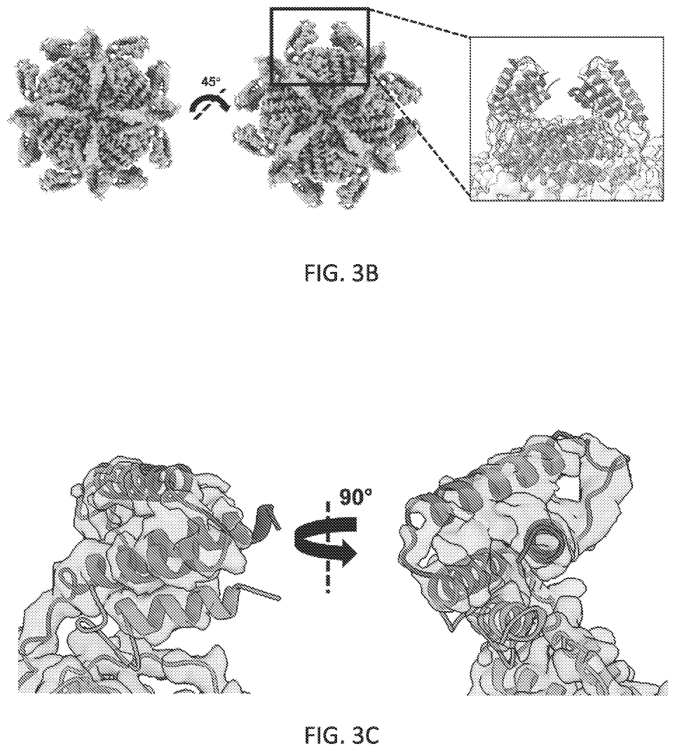

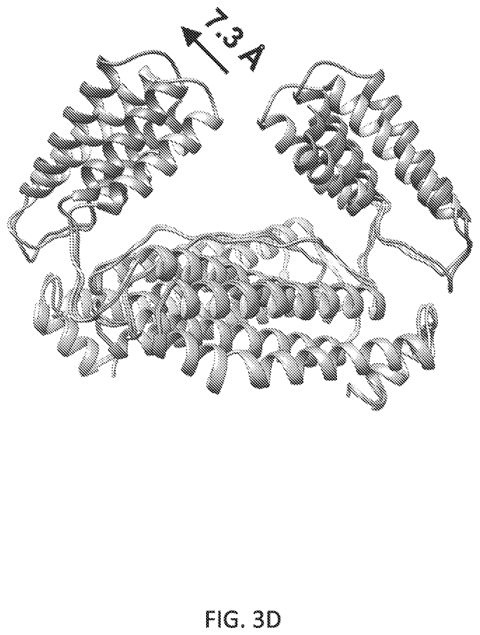

With the success of the structure determination of MKF, we further investigated whether the double-shell system can be applied to drug discovery for diseases like AML. The current treatments for AML are mainly chemotherapy or stem cell transplantation, which have strong side effects and are often unsuccessful. Hence, inhibiting the interaction between the pKID domain of CREB and the KIX domain may provide a more specific treatment for this disease, with fewer side effects. Unlike tyrosine kinases, the identification of small molecules to disrupt protein-protein interactions have traditionally been difficult due to large and relatively flat surface areas. To this end, we developed a novel approach to target the interaction between CBP and CREB. We used computational approaches to design 10 peptides to mimic pKID and act as competitive inhibitors of the pKID-KIX interaction (see “Methods”). We used molecular dynamics simulations to identify minimal segments of pKID, 16-19 amino acid residues in length, that were predicted to maintain key interaction hotspots. We also proposed sequence variations to explore these hotspots, and we capped both N- and C-termini with neutral groups to further stabilize the peptides (FIG. 3A).

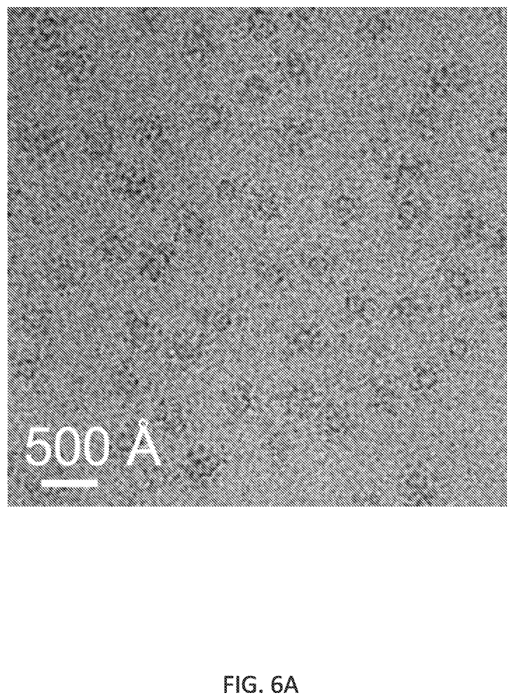

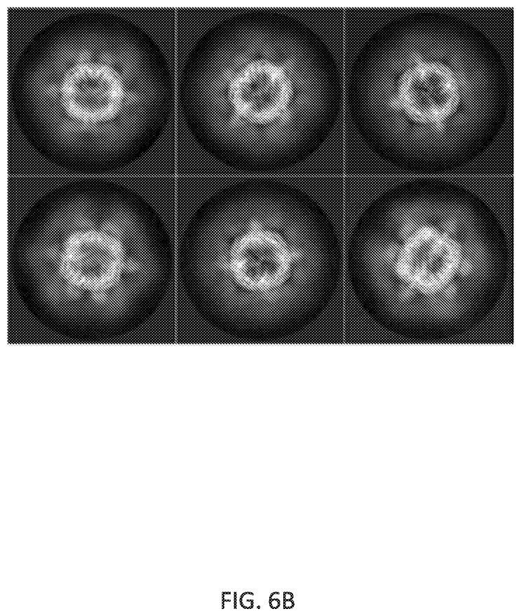

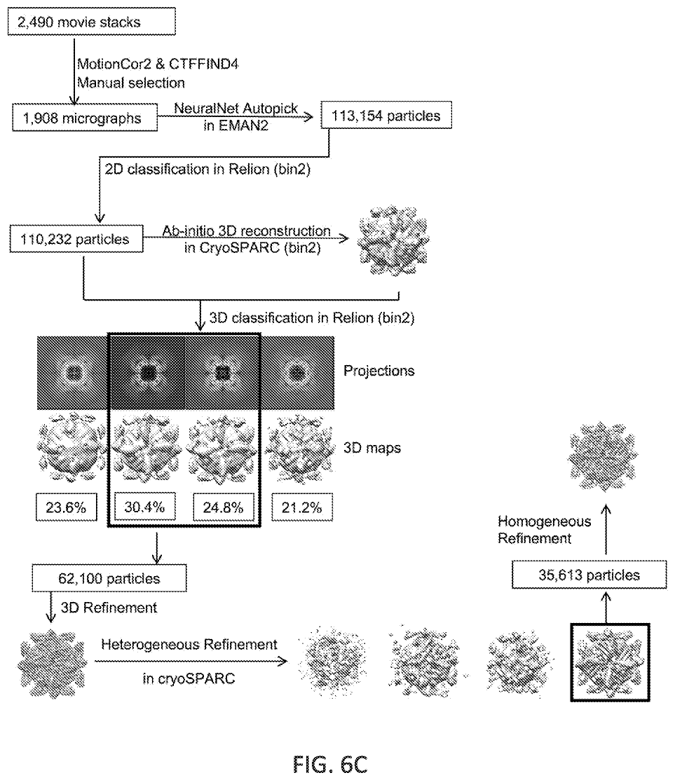

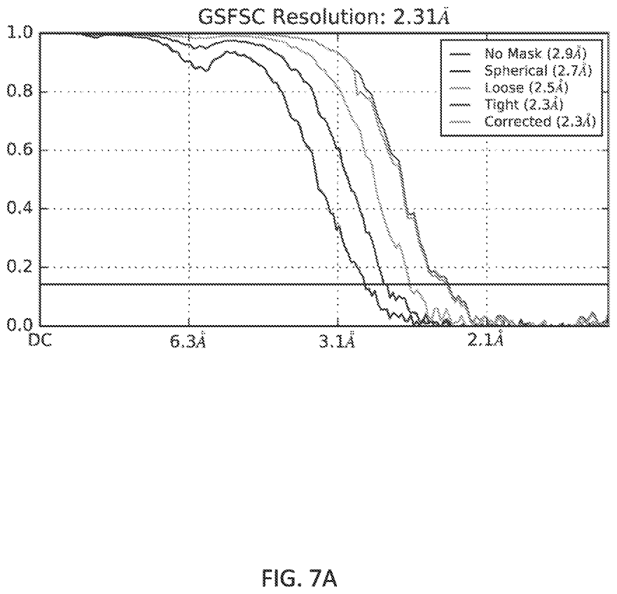

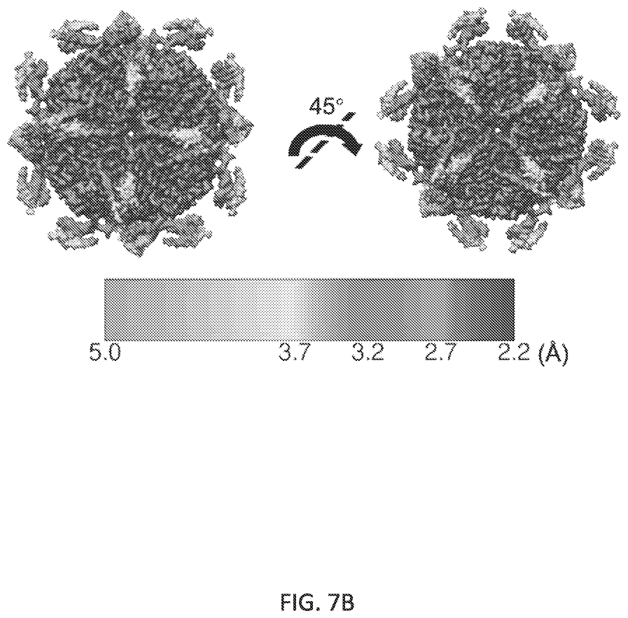

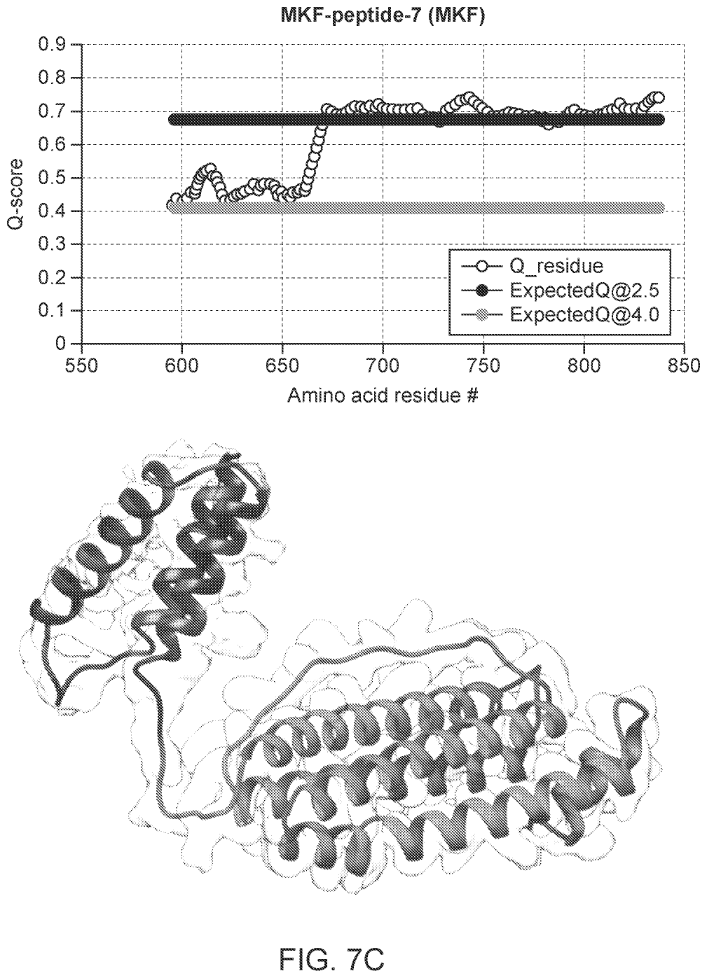

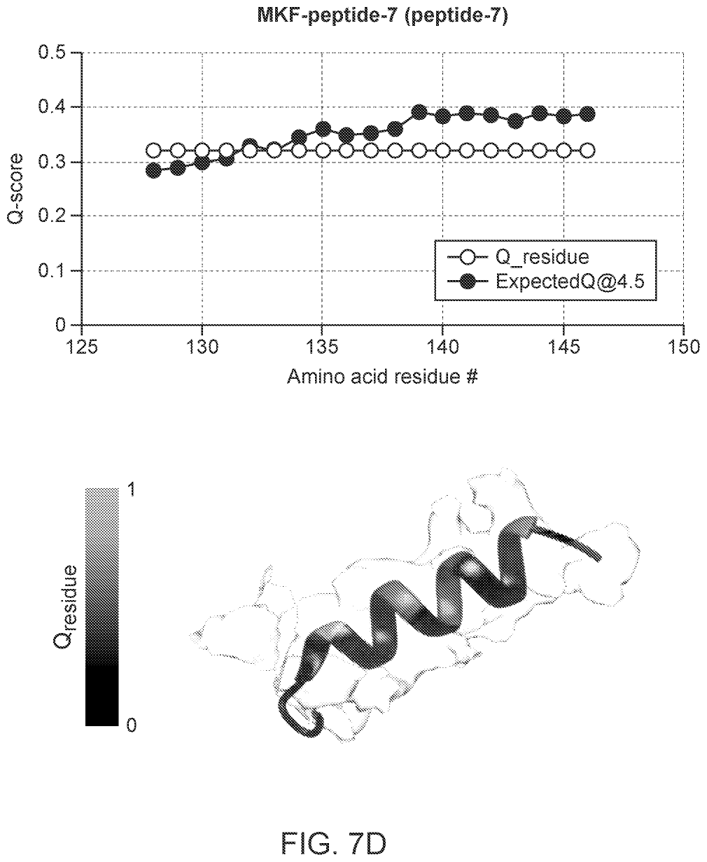

Among 10 computationally designed peptides, peptide-7 had the strongest in vitro binding affinity to KIX and was selected to form a complex with MKF, hereafter referred to as MKF-peptide-7. Using a similar single-particle cryo-EM processing strategy on this complex, we obtained a 3D reconstruction of the complex at an overall resolution of 2.3 Å (FIGS. 3B, 6, and 7 and Table S1). Similar to MKF, the apoferritin and KIX domain are well resolved while the MBP density is noisy (FIGS. 7B-7D). After modeling with apoferritin and KIX, we found an extra density bound to KIX, which should be derived from the peptide-7 (FIG. 3B), but we only can resolve its density at ˜4.5 Å (FIG. 3C, FIG. 7D). Then we used the particle symmetry expansion and focused classification options in the Relion software package¹⁷ for further image processing, aiming to improve the resolution for a single subunit. However, this strategy failed, possibly due to the disruption of disulfide bonds between one KIX and its neighboring apoferritin subunit. Compared to MKF, the KIX domain in the MKF-peptide-7 complex moves outward by 7.3 Å upon the binding of peptide-7 (FIG. 3D), suggesting that our double-shell system allows the movement of the cargo protein (KIX) to provide binding space for its partner (peptide-7) to avoid clashes with apoferritin. Together with the functional assay that shows the strong binding affinity of KIX for peptide-7, our results indicate that peptide-7 can be used as an initial candidate of KIX-pKID inhibitors for further improvements.

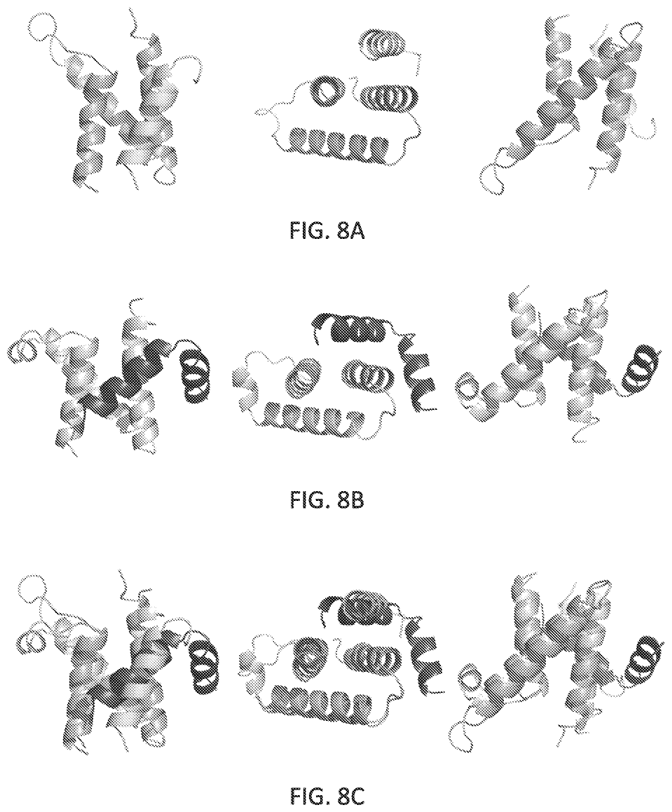

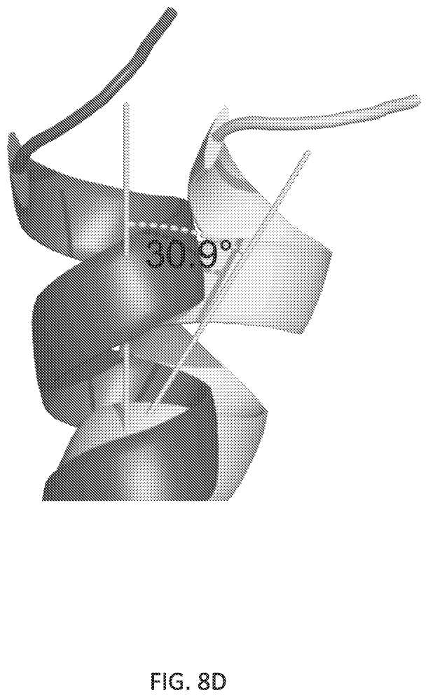

Interestingly, although our designed peptide is a truncated portion of the pKID helix that binds to KIX, we found that the binding mode of peptide-7 to KIX (FIG. 8A) differs from that of the corresponding region in pKID (PDB: 1 KDX¹⁸). In the previously published structure, the main alpha helix of pKID at the interaction surface contacts two helices of KIX, where hydrophobic residues pack into a pocket (FIG. 8B). In our structure, peptide-7 instead forms a helix-helix interaction with only a single KIX helix. Comparing the two structures, the angle of the helices from the extended pKID domain vs peptide-7 is offset by 30.9° (FIGS. 8C, 8D), further confirming the discovery of a unique binding mode of peptide-7 and KIX. This provides opportunities for further optimization with additional stabilization, for example, introducing staples across different parts of the helix.

The theoretical atomic resolution limit of 38-kDa for single-particle cryo-EM analysis (SPA) was proposed in 1995¹⁹. Until now, this limit has not yet been broken, pointing to the approximate truth that SPA is limited to samples below 38 kDa. In this study, we established the double-shell system, which can be applied in determining the structure of proteins as small as 11 kDa at near-atomic resolution, thereby broadening the application range of SPA. The 11-kDa KIX as the cargo protein in this work was resolved at near-atomic resolution, and its 19-residue peptide inhibitor was also detectable, suggesting that our double-shell double-shell system can also assist structure-based drug design. Though our structure does not yet resolve atomic details, the site of interaction between small molecules and the targeted protein is an important information to validate our design principle and to encourage the next phase of the drug development pipeline. In addition to interacting with pKID, the KIX domain also interacts with many other human and viral proteins, such as transcription factors p53 and FOXO3a, breast cancer type 1 susceptibility protein (BRCA1), and Tat protein of human immunodeficiency virus type 1 (HIV-1)²⁰. Therefore, the MKF design in this study can be applied to drug discovery for all these related diseases. Furthermore, since there is no specificity in this strategy, any small protein that is genetically fused to the double-shell system and rigidly displayed should be suitable for SPA, whether for solving the structure of small proteins or for drug discovery. Finally, the double-shell system may also play a role in vaccine development, e.g. fusion of the receptor-binding domain (RBD) of the spike protein of SARS-Cov-2 (COVID-19) to apoferritin could make them rigidly displayed on the apoferritin cage to mimic the virus and then be used as a potential vaccine²¹⁻²⁴.

Materials and Methods

Protein Expression and Purification

The KIX domain (588-672) of CBP and mouse apoferritin were grafted into the pMAL-c5X vector. To stabilize the KIX domain of CBP on apoferritin, five C-terminal amino acids of the KIX domain of CBP and five N-terminal amino acids of apoferritin were truncated from the construct. The site-directed mutagenesis was performed to introduce mutations in MBP-KIX-apoferritin (T27C, A31C, E169C, C177S, and E237C) (FIG. 4A). The MBP-KIX-apoferritin constructs were bacterially expressed in BL21 (DE3) and purified using amylose resin (New England Biolabs) with 20 mM maltose. The 24-mer MBP-KIX-apoferritin complex was further purified using HiLoad 16/600 Superdex 200 pg (Cytiva) from incomplete KIX-apoferritin complex and its monomer in 30 mM HEPES-NaOH (pH 7.5) containing 150 mM sodium chloride. Trehalose (final concentration: 5%) was added to the samples. For MBP-KIX-apoferritin in complex with the peptide 7, the purified MBP-KIX-apoferritin was mixed with the peptide 7 and stored at 4° C.

Computational Design of Therapeutic Peptides

The CREB protein is 340 amino acid residues in length and contains the pKID domain, which is 28 residues in length. To design a short peptide that would compete with pKID, we used molecular dynamics simulations to first study the interface between pKID and KIX to identify hotspot interactions. Hotspots were defined by measuring the highest frequency interactions between KIX and pKID (hydrophobic contacts, hydrogen bonds, salt bridges, and cation-pi interactions). We additionally performed simulations of truncated versions of the pKID domain that would maintain these interactions as well as several mutants. Simulation coordinates were prepared from the previously published pKID-KIX NMR structure¹⁸. Prime was used to model missing side chains, and neutral acetyl and methylamide groups were added to cap the protein termini. PropKa was used to determine the dominant protonation state of all titratable residues at pH 7. Dabble was used to place the protein complex into a water box (65×65×65 Å) containing sodium and chloride ions at roughly 150 mM²⁵. All simulations were run on a single Graphical Processing Unit (GPU) using the Amber18 Compute Unified Device Architecture (CUDA) version of particle-mesh Ewald molecular dynamics (PMEMD). We used the CHARMM36m parameters for proteins, sodium and chloride ions, and the TIP3P model for waters. Protocols for minimization, equilibration, and production are described in the previous publications²⁵⁻²⁶. All simulations were 1 μs in length. To analyze the stability of the peptide-KIX interaction, we calculated the root-mean-square fluctuation (RMSF) of the peptides after aligning the complex to the helical segments of the KIX domain. Using the calculated stabilities (based on RMSF) from these simulations, we selected the optimal peptide segment and most promising mutants to synthesize and test in vitro (FIG. 3A).

Fluorescence Polarization Assay

Fluorescence polarization (FP) using the MBP-KIX-apoferritin and the BODIPY-conjugated phosphorylated pKID domain of CREB was performed as described previously¹⁶. Briefly, the hexahistidine-tagged KID domain of CREB was bacterially expressed and purified using the TALON superflow (Cytiva). After the His-tag cleavage, the KID domain of CREB was further purified using HiLoad 16/600 Superdex 75 pg (Cytiva). The purified KID domain was enzymatically phosphorylated (pKID) by the catalytic subunit of the cAMP-dependent protein kinase A (PKA) (New England Biolabs). Phosphorylation efficiency of pKID, which was assessed using a SDS-gel containing Phos-tag™ Acrylamide (FUJIFILM), was almost 100%. pKID was purified using the phosphoprotein enrichment kit (Takara). BODIPY™ TMR C5-Maleimide (Thermo Fisher Scientific) was conjugated to the N-terminal cysteine residue of the purified pKID, and the unreacted BODIPY-dye was removed using the PD-10 column (Cytiva) twice. In the FP assay using 25 μl of 200 nM BODIPY-pKID was mixed with 25 μl of dilution series of KIX, MBP-KIX-apoferritin construct 3, and MBP-apoferritin (3.9 μM to 10 μM). Molar concentration of KIX, MBP-KIX-apoferritin construct 3, and MBP-apoferritin was calculated as a monomer. FP was measured 30 minutes after mixing using INFINITE M1000 PRO (TECAN). The experiment was performed 4 times, and the average was plotted with the standard deviation. The designed peptides were synthesized by Thermo Fisher Scientific. For the inhibition assay using the peptides, 25 μl of 400 nM each KIX and BODIPY-pKID was mixed with 25 μl of dilution series of peptides (122 nM to 2 mM). The experiment was performed 3 times, and the average was plotted with the standard deviation.

Cryo-EM Data Acquisition

The samples were diluted at a final concentration of around 0.5 mg/mL for both the MBP-KIX-apoferritin complex (MKF) and MKF-peptide-7 complex. Three microliters of the samples were applied onto glow-discharged 200-mesh R2/1 Quantifoil grids coated with continuous carbon. The grids were blotted for 2 s and rapidly cryocooled in liquid ethane using a Vitrobot Mark IV (Thermo Fisher Scientific) at 4° C. and 100% humidity. The samples were screened using a Talos Arctica cryo-electron microscope (Thermo Fisher Scientific) operated at 200 kV. They were then imaged in a Titan Krios cryo-electron microscope (Thermo Fisher Scientific) operated at 300 kV with GIF energy filter (Gatan) at a magnification of 165,000× (corresponding to a calibrated sampling of 0.82 Å per pixel) for both the samples. Micrographs were recorded by EPU software (Thermo Fisher Scientific) with a Gatan K2 Summit direct electron detector, where each image was composed of 30 individual frames with an exposure time of 6 s and an exposure rate of 7.3 electrons per second per Å². A total of 1,915 movie stacks for the MKF and 2,490 movie stacks for the MKF-peptide-7 complex were collected.

Single-Particle Image Processing and 3D Reconstruction

All micrographs were first imported into Relion¹⁷ for image processing. The motion-correction was performed using MotionCor2²⁷ and the contrast transfer function (CTF) was determined using CTFFIND4²⁸. Then the micrographs with “rlnMotionEarly <10” and “rlnCtfMaxResolution <5” were selected using the “subset selection” option in Relion. All particles were autopicked using the NeuralNet option in EMAN2²⁹. Then, particle coordinates were imported to Relion, where the poor 2D class averages were removed by several rounds of 2D classification. The initial models for both dataset were built in cryoSPARC³⁰ using ab-initio reconstruction option with octahedral symmetry applied separately. For the MKF, 167,662 particles were picked and 124,114 were selected after 2D classification in Relion. After removing bad classes by 3D classification, the 3D refinement was performed using 53,550 particles, and a 2.7 Å map was obtained, then heterogeneous refinement was applied in cryoSPARC to further clean up the particle images. Final homogeneous refinement was performed using 37,386 particles, and a 2.57 Å map was obtained. For the MKF-peptide-7, 113, 154 particles were picked and 110,232 were selected after 2D classification in Relion. After removing bad classes by 3D classification, the 3D refinement was performed using 62,100 particles, and a 2.51 Å map was obtained, then heterogeneous refinement was applied in cryoSPARC to further clean up the particles images. Final homogeneous refinement was performed using 35,613 particles, and a 2.3 Å map was obtained. Resolutions for the final maps were estimated using the 0.143 criterion of the Fourier shell correlation (FSC) curve without or with mask. A Gaussian low-pass filter was applied to the final 3D maps displayed in the UCSF Chimera software package³¹. (see more information in FIGS. 5, 6 and 7, and Table 1).

Model Building

Model building was first conducted on the cryo-EM map of the MBP-KIX-Ferritin complex (MKF). The MKF protomer consists of apoferritin and KIX. The crystal structures of apoferritin protomer (PDB ID: 3WNW) and KIX (PDB ID: 4190) were rigidly fitted into the cryo-EM map, followed by refinement using phenix.real_space_refine³² with secondary structure and geometry restraints. Coot³³ was applied to manually optimize the model. The protomer model was then fitted into the cryo-EM density of the other protomers with Chimera³⁴, and optimized using phenix.real_space_refine.

As the protomer was conformationally different in the cryo-EM density of the MKF-peptide-7 complex compared with MKF, molecular dynamics flexible fitting (MDFF)³⁵ was performed on the MKF-peptide-7 protomer using MKF protomer as the initial model. Each run of MDFF included 104 minimization steps and 105 molecular dynamics steps, and the MDFF was stopped after no noticeable structural changes. The resultant model was refined using phenix.real_space_refine. The NMR structure of pKID (residues 128-146 corresponding to 1-19 residues of peptide-7) was then rigidly docked into the extra density adjacent to KIX and optimized by Coot and phenix.real_space_refine. The protomer model was then fitted into the cryo-EM density of the other protomers with Chimera, and optimized using phenix.real_space_refine.

The final models were evaluated by MolProbity³⁶ as previously stated³⁷. Statistics of the map reconstruction and model building are summarized in Table 1. All figures were prepared using PyMol³⁸ and Chimera.

TABLE 1
Cryo-EM data collection, refinement and validation statistics

| | MKF | MKF-peptide-7 |
|---|---|---|
| | (EMDB-xxxx) | (EMDB-xxxx) |
| | (PDB xxxx) | (PDB xxxx) |
| Data collection and processing | | |
| Magnification | 165k | 165k |
| Voltage (kV) | 300 | 300 |
| Electron exposure (e−/Å²) | 7.3 | 7.3 |
| Defocus range (μm) | −0.9-−3.3 | −0.5-−2.9 |
| Pixel size (Å) | 0.82 | 0.82 |
| Symmetry imposed | ◯ | ◯ |
| Initial particle images (no.) | 167,662 | 113,154 |
| Final particle images (no.) | 37,386 | 35,613 |
| Map resolution (Å) | 2.57 | 2.31 |
| FSC threshold | 0.143 | 0.143 |
| Map resolution range (Å) | 2.2-5.0 | 2.2-5.0 |
| Refinement | | |
| Initial model used (PDB code) | | |
| Model resolution (Å) | 2.6 | 2.3 |
| FSC threshold | 0.143 | 0.143 |
| Model resolution range (Å) | 2.2-4.5 | 2.2-4.5 |
| Map sharpening B factor (Å²) | −10 (self-provided) | −10 (self-provided) |
| Model composition | | |
| Non-hydrogen atoms | 47,952 | 51,720 |
| Protein residues | 5,832 | 6,288 |
| Ligands | | |
| B factors (Å²) | | |
| Protein | 107.34 | 126 |
| Ligand | | |
| R.m.s. deviations | | |
| Bond lengths (Å) | 0.003 | 0.003 |
| Bond angles (°) | 0.791 | 0.752 |
| Validation | | |
| MolProbity score | 1.78 | 1.73 |
| Clashscore | 4.33 | 4.49 |
| Poor rotamers (%) | 1.06 | 0.43 |
| Ramachandran plot | | |
| Favored (%) | 90.13 | 91.37 |
| Allowed (%) | 9.46 | 7.84 |
| Disallowed (%) | 0.41 | 0.78 |

### REFERENCES

1. Herzik, M. A., Jr, Wu, M. & Lander, G. C. High-resolution structure determination of sub-100 kDa complexes using conventional cryo-EM. Nat. Commun. 10, 1032 (2019).
2. Khoshouei, M., Radjainia, M., Baumeister, W. & Danev, R. Cryo-EM structure of haemoglobin at 3.2 Å determined with the Volta phase plate. Nat. Commun. 8, 16099 (2017).
3. Fan, X. et al. Single particle cryo-EM reconstruction of 52 kDa streptavidin at 3.2 Angstrom resolution. Nat. Commun. 10, 2386 (2019).
4. Liu, Y., Gonen, S., Gonen, T. & Yeates, T. O. Near-atomic cryo-EM imaging of a small protein displayed on a designed scaffolding system. Proc. Natl. Acad. Sci. U.S.A 115, 3362-3367 (2018).
5. Liu, Y., Huynh, D. T. & Yeates, T. O. A 3.8 Å resolution cryo-EM structure of a small protein bound to an imaging scaffold. Nature Communications vol. 10 (2019).
6. Coscia, F. et al. Fusion to a homo-oligomeric scaffold allows cryo-EM analysis of a small protein. Sci. Rep. 6, 30909 (2016).
7. Yao, Q., Weaver, S. J., Mock, J.-Y. & Jensen, G. J. Fusion of DARPin to aldolase enables visualization of small protein by cryoEM. doi:10.1101/455063.
8. Zhang, K. et al. Cryo-EM structure of a 40 kDa SAM-IV riboswitch RNA at 3.7 Å resolution. Nat. Commun. 10, 5511 (2019).
9. Zhang, K. et al. Structure of the 30 kDa HIV-1 RNA Dimerization Signal by a Hybrid Cryo-EM, NMR, and Molecular Dynamics Approach. Structure 26, 490-498. e3 (2018).
10. Nakane, T. et al. Single-particle cryo-EM at atomic resolution. 2020.05.22.110189 (2020) doi:10.1101/2020.05.22.110189.
11. Yip, K. M., Fischer, N., Paknia, E., Chari, A. & Stark, H. Breaking the next Cryo-EM resolution barrier—Atomic resolution determination of proteins! 2020.05.21.106740 (2020) doi:10.1101/2020.05.21.106740.
12. Zhang, K., Pintilie, G. D., Li, S., Schmid, M. F. & Chiu, W. Resolving Individual-Atom of Protein Complex using Commonly Available 300-kV Cryo-electron Microscopes. 2020.08.19.256909 (2020) doi:10.1101/2020.08.19.256909.
13. Shankar, D. B. et al. The role of CREB as a proto-oncogene in hematopoiesis and in acute myeloid leukemia. Cancer Cell 7, 351-362 (2005).
14. Mitton, B. et al. Small molecule inhibition of cAMP response element binding protein in human acute myeloid leukemia cells. Leukemia 30, 2302-2311 (2016).
15. Wang, N. et al. Ordering a Dynamic Protein Via a Small-Molecule Stabilizer. Journal of the American Chemical Society vol. 135 3363-3366 (2013).
16. Chae, H.-D. et al. SAR optimization studies on modified salicylamides as a potential treatment for acute myeloid leukemia through inhibition of the CREB pathway. Bioorg. Med. Chem. Lett. 29, 2307-2315 (2019).
18. Scheres, S. H. W. RELION: implementation of a Bayesian approach to cryo-EM structure determination. J. Struct. Biol. 180, 519-530 (2012).
18. Radhakrishnan, I. et al. Solution structure of the KIX domain of CBP bound to the transactivation domain of CREB: a model for activator:coactivator interactions. Cell 91, 741-752 (1997).
19. Henderson, R. The potential and limitations of neutrons, electrons and X-rays for atomic resolution microscopy of unstained biological molecules. Q. Rev. Biophys. 28, 171-193 (1995).
20. Thakur, J. K., Yadav, A. & Yadav, G. Molecular recognition by the KIX domain and its role in gene regulation. Nucleic Acids Res. 42, 2112-2125 (2014).
21. Bianchi, M. et al. Electron-Microscopy-Based Epitope Mapping Defines Specificities of Polyclonal Antibodies Elicited during HIV-1 BG505 Envelope Trimer Immunization. Immunity 49, 288-300. e8 (2018).
22. Al-Halifa, S., Gauthier, L., Arpin, D., Bourgault, S. & Archambault, D. Nanoparticle-Based Vaccines Against Respiratory Viruses. Front. Immunol. 10, 22 (2019).
23. Reljic, R. & Gonzdlez-Ferndndez, Á. Editorial: Nanoparticle Vaccines Against Infectious Diseases. Front. Immunol. 10, 2615 (2019).
24. Wang, Z. et al. Functional ferritin nanoparticles for biomedical applications. Front Chem Sci Eng 11, 633-646 (2017).
25. Sicilia, M.-A., Garcia-Barriocanal, E. & Sánchez-Alonso, S. Community Curation in Open Dataset Repositories: Insights from Zenodo. Procedia Computer Science vol. 106 54-60 (2017).
26. Latorraca, N. R. et al. Molecular mechanism of GPCR-mediated arrestin activation. Nature 557, 452-456 (2018).
27. Zheng, S. Q. et al. MotionCor2: anisotropic correction of beam-induced motion for improved cryo-electron microscopy. Nat. Methods 14, 331-332 (2017).
28. Rohou, A. & Grigorieff, N. CTFFIND4: Fast and accurate defocus estimation from electron micrographs. J. Struct. Biol. 192, 216-221 (2015).
29. Tang, G. et al. EMAN2: an extensible image processing suite for electron microscopy. J. Struct. Biol. 157, 38-46 (2007).
30. Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
31. Pettersen, E. F. et al. UCSF Chimera—a visualization system for exploratory research and analysis. J. Comput. Chem. 25, 1605-1612 (2004).
32. Adams, P. D. et al. PHENIX: a comprehensive Python-based system for macromolecular structure solution. in International Tables for Crystallography 539-547 (2012).
33. Emsley, P., Lohkamp, B., Scott, W. G. & Cowtan, K. Features and development of Coot. Acta Crystallogr. D Biol. Crystallogr. 66, 486-501 (2010).
34. Pettersen, E. F. et al. UCSF Chimera—a visualization system for exploratory research and analysis. J. Comput. Chem. 25, 1605-1612 (2004).
35. Trabuco, L. G., Villa, E., Mitra, K., Frank, J. & Schulten, K. Flexible fitting of atomic structures into electron microscopy maps using molecular dynamics. Structure 16, 673-683 (2008).
36. Chen, V. B. et al. MolProbity: all-atom structure validation for macromolecular crystallography. Acta Crystallogr. D Biol. Crystallogr. 66, 12-21 (2010).
37. Zhang, K. et al. Inhibition mechanisms of AcrF9, AcrF8, and AcrF6 against type I-F CRISPR-Cas complex revealed by cryo-EM. Proceedings of the National Academy of Sciences vol. 117 7176-7182 (2020).
38. Rigsby, R. E. & Parker, A. B. Using the PyMOL application to reinforce visual understanding of protein structure. Biochem. Mol. Biol. Educ. 44, 433-437 (2016).

What is claimed is:

1. A protein double-shell nanostructure comprising:
a) an inner shell comprising a plurality of apoferritin proteins;
b) a cargo protein of interest, wherein the cargo protein is connected to the N-terminus of the apoferritin; and
c) an outer shell comprising a tag protein, wherein the tag protein is connected to the cargo protein of interest such that the tag protein points outward from the inner shell and increases rigidity of the cargo protein of interest.

2. The protein double-shell nanostructure of claim 1, wherein the inner shell consists of 24 apoferritin proteins.

3. The protein double-shell nanostructure of claim 1 or 2, wherein the cargo protein of interest is smaller than 50 kilodaltons (kDa).

4. The protein double-shell nanostructure of claim 3, wherein the cargo protein of interest ranges in size from 11 kDa to 50 kDa.

5. The protein double-shell nanostructure of any one of claims 1-4, wherein the tag protein is a maltose-binding protein (MBP).

6. The protein double-shell nanostructure of any one of claims 1-5, wherein the apoferritin protein is a truncated apoferritin protein lacking up to the first five N-terminal amino acids residues of SEQ ID NO:1.

7. The protein double-shell nanostructure of any one of claims 1-6, wherein the apoferritin protein is a truncated apoferritin protein consisting of amino acids 6 to 181 of apoferritin numbered relative to the reference sequence of SEQ ID NO:1.

8. The protein double-shell nanostructure of any one of claims 1-7, wherein the apoferritin comprises a substitution of a cysteine at a position corresponding to D93, E95, and E163 of the apoferritin numbered relative to the reference sequence of SEQ ID NO:1.

9. The protein double-shell nanostructure of claim 8, wherein the apoferritin further comprises a substitution of a serine at a position corresponding to C103 of the apoferritin numbered relative to the reference sequence of SEQ ID NO:1.

10. The protein double-shell nanostructure of claim 8 or 9, further comprising a disulfide bond between a cysteine of the apoferritin and a cysteine of the cargo protein.

11. The protein double-shell nanostructure of claim 10, wherein the cysteine of the apoferritin or the cysteine of the cargo protein is a naturally occurring cysteine or an engineered cysteine mutation.

12. The protein double-shell nanostructure of any one of claims 1-11, wherein the cargo protein of interest comprises a KIX domain.

13. The protein double-shell nanostructure of claim 12, wherein the KIX domain is a truncated KIX domain consisting of amino acids 1-80 of the KIX domain numbered relative to the reference sequence of SEQ ID NO:2.

14. The protein double-shell nanostructure of claim 12 or 13, wherein the KIX domain comprises a substitution of a cysteine at a position corresponding to T27 and A31 of the KIX domain numbered relative to the reference sequence of SEQ ID NO:2.

15. The protein double-shell nanostructure of any one of claims 1-14, wherein the cargo protein retains biological activity within the protein double-shell nanostructure.

16. The protein double-shell nanostructure of any one of claims 1-15, further comprising a linker.

17. The protein double-shell nanostructure of claim 16, wherein the linker is between the cargo protein and the tag protein.

18. The protein double-shell nanostructure of claim 16 or 17, wherein the linker is between the cargo protein and the apoferritin.

19. A complex comprising the protein double-shell nanostructure of any one of claims 1-18 and a binding agent, wherein the binding agent binds to the cargo protein within the protein double-shell nanostructure.

20. The complex of claim 19, wherein the binding agent is a substrate, inhibitor, agonist, antagonist, or ligand of the cargo protein.

21. A method of performing single-particle cryogenic-electron microscopy (cryo-EM) using the protein double-shell nanostructure of any one of claims 1-18 to determine the structure of the cargo protein of interest, the method comprising:
a) providing the protein double-shell nanostructure of any one of claims 1-18; and
b) performing single-particle cryo-EM on the protein double-shell nanostructure to determine the structure of the cargo protein within the protein double-shell nanostructure.

22. The method of claim 21, further comprising contacting the protein double-shell nanostructure with a binding agent prior to said performing single-particle cryo-EM on the protein double-shell nanostructure, wherein the binding agent binds to the cargo protein within the protein double-shell nanostructure, wherein said performing single-particle cryo-EM comprises performing cryo-EM on a complex of the protein double-shell nanostructure with the binding agent.

23. The method of claim 21 or 22, further comprising using the cryo-EM structure of the cargo protein to identify a small molecule that binds to the cargo protein, the method comprising: a) screening in silico a small molecule library for candidate small molecules likely to bind to the cargo protein using a three-dimensional model of the cargo protein that is computationally derived from the atomic coordinates of the cryo-EM structure of the cargo protein; and b) evaluating the candidate small molecules identified in step (a) as likely to bind to the cargo protein for their ability to effect activity of the cargo protein using one or more in vitro or in vivo assays to identify at least one candidate small molecule that inhibits or activates activity of the cargo protein.

24. A fusion protein comprising:
a) an apoferritin protein;
b) a cargo protein of interest, wherein the cargo protein is connected to the N-terminus of the apoferritin; and
c) a tag protein, wherein the tag protein is connected to the cargo protein of interest.

25. A vector comprising an expression cassette for expressing the fusion protein of claim 24.

26. The vector of claim 25, wherein the vector is a non-viral or a viral vector.

27. The vector of claim 25 or 26, wherein the expression cassette comprises a promoter operably linked to a coding sequence encoding the fusion protein.

28. The vector of claim 25 or 26, wherein the expression cassette comprises:
a) a coding sequence encoding the tag protein;
b) a coding sequence encoding the apoferritin protein; and
c) a multiple cloning site for insertion of a coding sequence encoding the cargo protein of interest in-frame between the coding sequence encoding the tag protein and the coding sequence encoding the apoferritin protein.

29. The vector of claim 25 or 26, wherein the expression cassette comprises a multiple cloning site for insertion of a coding sequence encoding the fusion protein.

30. A method of producing a protein double-shell nanostructure, the method comprising:
c) transfecting a host cell with the vector of any one of claims 25-29; and
d) culturing the host cell under conditions suitable for expression of the fusion protein from the vector, wherein the fusion protein assembles into the protein double-shell nanostructure.

31. A kit comprising the protein double-shell nanostructure of any one of claims 1-18.

32. A kit comprising the fusion protein of claim 24.

33. A kit comprising the vector of any one of claims 25-29.


SEQUENCE LISTING (15 sequences)

SEQ ID NO: 1 - protein, 182 aa, Mus musculus
  ref <308>: DATABASE ACCESSION NUMBER: Genpept/6SHT_A
  ref <309>: DATABASE ENTRY DATE: 2020-12-01
  ref <313>: RELEVANT RESIDUES IN SEQ ID NO: (1)..(182)
MTTASPSQVRQNYHQDAEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDES

SEQ ID NO: 2 - protein, 80 aa, Mus musculus
RKGWHEHVTQDLRSHLVHKLVQAIFPTPDPAALKDRRMENLVAYAKKVEGDMYESANSRDEYYHLLAEKIYKIQKELEEK

SEQ ID NO: 3 - protein, 212 aa, Mus musculus
  ref <308>: DATABASE ACCESSION NUMBER: PDB/3WNW_L
  ref <309>: DATABASE ENTRY DATE: 2020-12-01
  ref <313>: RELEVANT RESIDUES IN SEQ ID NO: (1)..(212)
GSHMASMTGGQQMGRGSMTTASPSQVRQNYHQDAEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDESGDIESAQSDEEVE

SEQ ID NO: 4 - protein, 81 aa, Mus musculus
  misc_feature (1)..(1): Xaa can be any naturally occurring amino acid
  ref <308>: DATABASE ACCESSION NUMBER: PDB/1KDX_A
  ref <309>: DATABASE ENTRY DATE: 2020-12-01
  ref <313>: RELEVANT RESIDUES IN SEQ ID NO: (1)..(81)
XVRKGWHEHVTQDLRSHLVHKLVQAIFPTPDPAALKDRRMENLVAYAKKVEGDMYESANSRDEYYHLLAEKIYKIQKELEE

SEQ ID NO: 5 - protein, 101 aa, Mus musculus
  misc_feature (1)..(1): Xaa can be any naturally occurring amino acid
  misc_feature (12)..(12): Xaa can be any naturally occurring amino acid
  misc_feature (54)..(54): Xaa can be any naturally occurring amino acid
  misc_feature (68)..(68): Xaa can be any naturally occurring amino acid
  ref <308>: DATABASE ACCESSION NUMBER: PDB.4I9O_A
  ref <309>: DATABASE ENTRY DATE: 2020-12-01
  ref <313>: RELEVANT RESIDUES IN SEQ ID NO: (1)..(101)
XRGSHHHHHHGXASGVRKGWHEHVTQDLRSHLVHKLVQAIFPTPDPAALKDRRXENLVAYAKKVEGDXYESANSRDEYYHLLAEKIYKIQKECEEKRRSRL

SEQ ID NO: 6 - protein, 17 aa, Artificial Sequence
  peptide 1
RPPSYRKILNDLSSDAP

SEQ ID NO: 7 - protein, 17 aa, Artificial Sequence
  peptide 2
RPPSYRKILNDFSSDAP

SEQ ID NO: 8 - protein, 17 aa, Artificial Sequence
  peptide 3
RPPSYRKILNDISSDAP

SEQ ID NO: 9 - protein, 19 aa, Artificial Sequence
  peptide 4
RPPSYRKCHALNDISSDAP

SEQ ID NO: 10 - protein, 16 aa, Artificial Sequence
  peptide 5
RPPSYRKILNDLSSDA

SEQ ID NO: 11 - protein, 17 aa, Artificial Sequence
  peptide 6
RPPSYRKILEDLSSDAP

SEQ ID NO: 12 - protein, 20 aa, Artificial Sequence
  peptide 7
LSRRPPSYRKILNDLSSDAP

SEQ ID NO: 13 - protein, 15 aa, Artificial Sequence
  peptide 8
PEYRKILNDLSSDAP

SEQ ID NO: 14 - protein, 12 aa, Artificial Sequence
  peptide 9
EYRKILNDLSSD

SEQ ID NO: 15 - protein, 18 aa, Artificial Sequence
  peptide 10
KRIKELELLLMSTENELK